(19) United States
(12) Patent Application Publication
(10) Pub. No.: US 2006/0035829 A1
(43) Pub. Date: Feb. 16, 2006

(54) Chemokine combinations to mobilize progenitor/stem cells

(75) Inventors: Gary J. Bridger, Bellingham, WA (US); Louis M. Pelus, Indianapolis, IN (US)

Correspondence Address: MORRISON & FOERSTER LLP, 12531 HIGH BLUFF DRIVE, SUITE 100, SAN DIEGO, CA, 92130-2040, US

(73) Assignee: AnorMED Inc.

(21) Appl. No.: 11/202,482

(22) Filed: Aug. 11, 2005

Related U.S. Application Data
(60) Provisional application No. 60/601,367, filed on Aug. 13, 2004.

(51) Int. Cl. A61K 38/19; A61K 31/33

(52) U.S. Cl. 514/12; 514/183

(57) ABSTRACT

Methods to elevate progenitor and stem cell counts in animal subjects using compounds which bind to the chemokine receptor CXCR4 in combination with the CXCR2 chemokine GROβ, including its modified forms, are disclosed.

### CROSS-REFERENCE TO RELATED APPLICATIONS

This application claims priority to U.S. provisional patent application Ser. No. 60/601,367, filed 13 Aug. 2004, which is incorporated herein by reference in its entirety.

### TECHNICAL FIELD

The invention is in the field of therapeutics and medicinal chemistry. More particularly, the invention concerns methods to mobilize progenitor/stem cells using combination therapy.

### BACKGROUND ART

Blood cells play a crucial part in maintaining the health and viability of animals, including humans. White blood cells include neutrophils, macrophage, eosinophils and basophils/mast cells, as well the B and T cells of the immune system. White blood cells are continuously replaced via the hematopoietic system, by the action of colony stimulating factors (CSF), and various cytokines on stem cells and progenitor cells in hematopoietic tissues. The nucleotide sequences encoding a number of these growth factors have been cloned and sequenced. Perhaps the most widely known of these is granulocyte colony stimulating factor (G-CSF), which has been approved for use in counteracting the negative effects of chemotherapy by stimulating the production of white blood cells and progenitor cells (peripheral blood stem cell mobilization). A discussion of the hematopoietic effects of this factor can be found, for example, in U.S. Pat. No. 5,582,823, incorporated herein by reference.

Several other factors have been reported to increase white blood cells and progenitor cells in both human and animal subjects. These agents include granulocyte-macrophage colony stimulating factor (GM-CSF), Interleukin-1 (IL-1), Interleukin-3 (IL-3), Interleukin-8 (IL-8), PIXY-321 (GM-CSF/IL-3 fusion protein), macrophage inflammatory protein, stem cell factor (SCF), thrombopoietin, flt-3, myelopoietin, anti-VLA4 antibody, and growth related oncogene (GRO), as single agents or in combination (Dale, D., et al., Am. J. of Hematol. (1998) 57:7-15; Rosenfeld, C., et al., Bone Marrow Transplantation (1997) 17:179-183; Pruijt, J., et al., Cur. Op. in Hematol. (1999) 6:152-158; Broxmeyer, H., et al., Exp. Hematol. (1995) 23:335-340; Broxmeyer, et al., Blood Cells, Molecules and Diseases (1998) 24:14-30; Glaspy, J., et al., Cancer Chemother. Pharmacol. (1996) 38 (suppl): S53-S57; Vadhan-Raj, S., et al., Ann. Intern. Med. (1997) 126:673-681; King, A., et al., Blood (2001) 97:1534-1542; Glaspy, J., et al., Blood (1997) 90:2939-2951).

Furthermore, King, et al. (King, A., et al., Blood (2001) 97:1534-1542) have demonstrated that a recombinant N-terminal 4-amino acid truncated form of the human chemokine GROβ (also known as SB-251353 or Garnocestim) can mobilize progenitor cells after administration of SB-251353 in combination with G-CSF where neutrophils and platelets were mobilized during the studies. Chemokines such as the SB-251353, GROα, GROβ, and GROγ are further discussed in WO 94/29341; WO 97/15594; WO 97/15595; WO 99/26645; WO 02/02132; U.S. Pat. No. 6,080,398; U.S. Pat. No. 6,399,053; and U.S. Pat. No. 6,447,766, all incorporated herein by reference.

SB-251353 is a basic, heparin-binding protein with a molecular mass of approximately 7500 Da, and is a specific CXCR2 receptor agonist (King, A., et al., J. Immunol. (2000) 164: 3774-3782, Hepburn, T., et al., Journal of Pharmacology and Experimental Therapeutics, (2001) 298: 886-893). Other chemokines, in addition to GROβ, acting via the CXCR2 receptor include GROα, GROγ, GCP-2 (granulocyte chemo-attractant protein 2), IL-8, NAP-2 (neutrophil activating peptide 2), ENA-78 (epithelial-cell derived neutrophil activating protein 78), and MGSA.

The chemokine receptor CXCR4 and its natural ligand stromal cell derived factor-1 (SDF-1) appear to be important in the process of development and maturation of blood cells, wherein mature blood cells are derived from hematopoietic precursor cells (progenitor) cells and stem cells present in specific hematopoietic tissues including bone marrow (for reviews see Maekawa, T., et al., Internal Med. (2000) 39:90-100; Nagasawa, T., et al., Int. J. Hematol. (2000) 72:408-411). This is demonstrated by reports that CXCR4 or SDF-1 knock-out mice exhibit hematopoietic defects (Ma, Q., et al., Proc. Natl. Acad. Sci USA (1998) 95:9448-9453; Tachibana, K., et al., Nature (1998) 393:591-594; Zou, Y-R., et al., Nature (1998) 393:595-599). It is also known that CD34+ progenitor cells express CXCR4 and require SDF-1 produced by bone marrow stromal cells for chemoattraction and engraftment (Peled, A., et al., Science (1999) 283:845-848) and that in vitro, SDF-I is chemotactic for both CD34+ cells (Aiuti, A., et al., J. Exp. Med. (1997) 185:111-120; Viardot, A., et al., Ann. Hematol. (1998) 77:194-197) and for progenitor/stem cells (Jo, D-Y., et al., J. Clin. Invest. (2000) 105:101-111). SDF-1 is also an important chemoattractant, signaling via the CXCR4 receptor, for several other more committed progenitors and mature blood cells including T-lymphocytes and monocytes (Bleul, C., et al., J. Exp. Med. (1996) 184:1101-1109), pro-and pre-B lymphocytes (Fedyk, E. R., et al., J. Leukoc. Biol. (1999) 66:667-673; Ma, Q., et al., Immunity (1999) 10:463-471) and megakaryocytes (Hodohara, K., et al., Blood (2000) 95:769-775; Riviere, C., et al., Blood (1999) 95:1511-1523; Majka, M., et al., Blood (2000) 96:4142-4151; Gear, A., et al., Blood (2001) 97:937-945; Abi-Younes, S., et al., Circ. Res. (2000) 86:131-138).

Thus, in summary, it appears that SDF-I is able to control the positioning and differentiation of cells bearing CXCR4 receptors, whether these cells are stem cells (i.e., cells which are CD34+) or progenitor cells (which result in formation of specified types of colonies in response to particular stimuli; that can be CD34⁺ or CD34⁻), or cells that are somewhat more differentiated.

Recently, considerable attention has been focused on the number of CD34+ cells mobilized in the pool of peripheral blood progenitor cells used for autologous stem cell transplantation. Stem cell transplantation can be characterized as either allogenic, where cells are transplanted from a healthy donor, usually a sibling, or as autologous, where cells are collected from the patient and reinfused after chemotherapy. During a typical procedure to collect stem cells, patient or donor receives a daily dose of G-CSF, for four or five consecutive days to stimulate stem cell production with apheresis occurring on following days until a target level of cells is reached. G-CSF use is also continued on apheresis days. It is common where significant number of patients do require multiple apheresis sessions in order to reach the target level of cells. The CD34+ population is the component thought to be primarily responsible for the improved recovery time after chemotherapy, and the cells most likely responsible for long-term engraftment and restoration of hematopoiesis (Croop, J. M., et al., Bone Marrow Transplantation (2000) 26:1271-1279). The mechanism by which CD34+ cells re-engraft may be due to the chemotactic effects of SDF-1 on CXCR4 expressing cells (Voermans, C., Blood (2001) 97:799-804; Ponomaryov, T., et al., J. Clin. Invest. (2000) 106:1331-1339). More recently, adult hematopoietic stem cells were shown to be capable of restoring damaged cardiac tissue in mice (Jackson, K., et al., J. Clin. Invest. (2001) 107:1395-1402; Kocher, A., et al., Nature Med. (2001) 7:430-436).

Thus, the role of the CXCR4 receptor in managing cell positioning and differentiation has assumed considerable significance. The compound AMD3100, which is 1,1[1,4-phenylene-bis(methylene)]-bis-1,4,8,11-tetraazacyclotetradecane, is a known CXCR4 antagonist which itself mobilizes progenitor cells (see, for example, Hubel, K., et al., Supportive Cancer Therapy (2004) 1:165-172, citing De Clercq, E., et al., Nat. Rev. Drug Discov. (2003) 2:581-587. In addition, PCT publication WO 00/45814 discloses that various cyclic polyamine compounds, including AMD3100 elevate white blood cell counts. PCT publication WO 03/011277 further shows that such compounds, including AMD3100, mobilize progenitor/stem cells; a combination of AMD3100 with various other factors, including GM-CSF, IL-1, IL-3, IL-8, PIXY-321 macrophage inflammatory protein, skin cell factor, thrombopoietin, growth-related oncogene or chemotherapy, or additional active ingredients generally, such as antibiotics, vitamins, herbal extracts, anti-inflammatories, glucose, anti-pyretics, analgesics is also mentioned.

It has now been found that the specific combination of a CXCR4 antagonist with GROβ, including the modified forms of GROβ, is particularly effective in mobilizing progenitor cells.

Citation of the above documents is not intended as an admission that any of the foregoing is pertinent prior art. All statements as to the date or representation as to the contents of these documents is based on the information available to the applicants and does not constitute any admission as to the correctness of the dates or contents of these documents. Further, all documents referred to throughout this application are incorporated in their entirety by reference herein.

### DISCLOSURE OF THE INVENTION

The invention is directed to methods of treating animal subjects, in particular, veterinary and human subjects, to enhance the number of progenitor cells and/or stem cells. The progenitor and/or stem cells may be harvested and used in cell transplantation. The methods of the invention employ inhibitors of the CXCR4 receptor such as certain polyamines described below in combination with at least one form of GROβ, i.e., GROβ itself or a modified form thereof. The methods are useful in the context of stem cell transplantation, tissue repair, and in situations where direct in vivo stimulation of hematopoiesis is desirable.

As used herein, the terms “a,” “an,” and “the” encompass singular and plural references, unless the context clearly dictates otherwise. Thus, for example, references to a CXCR4 antagonist encompasses or or more CXCR4 antagonist.

In one aspect, therefore, the invention is directed to a method to elevate the progenitor cells and/or stem cells, in a subject, which method comprises administering to said subject an amount of a compound that inhibits the CXCR4 receptor, such as that of formula (1) shown below, in combination with a member of collection represented by GROβ and modified forms thereof. In one embodiment, bone marrow cells are mobilized for myocardial repair. Other embodiments include mobilization of cells ex vivo or in vitro for subsequent transplantation into autologous or allogenic subjects.

Thus the methods of the invention also include treatment of cell populations ex vivo with combinations of a CXCR4 inhibitor and GROβ or its modified forms and introducing the treated populations into a compatible subject to enhance the population of stem cells and/or progenitor cells in the peripheral blood. An enhanced production of white blood cells in the bone marrow may result as well.

In additional aspects, the invention is directed to a combination product comprising a CXCR4 antagonist and a GROβ protein, wherein the combination is capable of elevating progenitor and/or stem cell population in peripheral blood or bone marrow. The combination product may be a mixture, a solution, or a pharmaceutical composition. In another aspect, the invention is directed to pharmaceutical compositions containing a CXCR4 inhibitor and a GROβ chemokine for use in effecting an elevation of progenitor cells and/or stem cells in animal subjects or in ex vivo cultures.

Furthermore, the present invention provides for the use of a combination product comprising a CXCR4 antagonist and a GROβ protein, or pharmaceutical compositions thereof, for elevating progenitor and/or stem cell population in peripheral blood or bone marrow. The present invention also provides for the use of a combination product comprising a CXCR4 antagonist and a GROβ protein, or pharmaceutical compositions thereof, for the manufacture of a medicament for elevating progenitor and/or stem cell population in peripheral blood or bone marrow. Further, the present invention is directed to compounds or drugs comprising a CXCR4 antagonist and a GROβ protein, for elevating progenitor and/or stem cell population in peripheral blood or bone marrow.

Surprisingly, the combination of a CXCR4 antagonist, such as a compound of formula (1) along with the chemokine GROβ, including a modified form thereof, is able to effect a marked mobilization of progenitor and stem cells in a short time—in less than a hour, as opposed to hours or days as would be required by either agent alone.

The CXCR4 antagonists for use in the methods of the present invention are exemplified by compounds of formula (1) of the formula:
Z-linker-Z′ (1)
wherein Z is an optionally substituted cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
or Z is of the formula 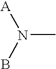
wherein A comprises a monocyclic or bicyclic fused ring system containing at least one N and B is H or an organic moiety of 1-20 atoms;
Z′ may be embodied in a form as defined by Z above, or alternatively may be of the formula
   —N(R)—(CR₂)ₙ—X
wherein each R is independently H or straight, branched or cyclic alkyl (1-6C),
n is 1 or 2, and
X is an aromatic ring, including heteroaromatic rings, or is a mercaptan;
or wherein Z′ can be a nitrogen-containing heterocycle, or can be NR₂ where each R is as defined above;
“linker” represents a bond, alkylene (1-6C) or may comprise aryl, fused aryl, oxygen atoms contained in an alkylene chain, or may contain keto groups or nitrogen or sulfur atoms.

The CXCR4 antagonists are preferably compounds having formula (1), as described above. The invention is also directed to a method to elevate the progenitor cells and/or stem cells, in a subject, comprising administering to said subject an amount of at least one compound that inhibits the CXCR4 receptor, such as that of formula (1A)-(1F), (2A)-(2B), (3), (3A)-(3C), (4), (4A)-(4C), (5), (6), (6A)-(6D), (7) and (8) as shown below, in combination with at least one member of collection represented by GROβ and modified forms thereof.

### MODES OF CARRYING OUT THE INVENTION

The invention relates to the specific combination of a CXCR4 antagonist with a GROβ protein to mobilize or enhance the proliferation of progenitor and/or stem cells. The combination is able to accomplish this stimulation in a much shorter time than either component alone and in a much shorter time than previously disclosed combinations. Mobilization of stem cells and/or progenitor cells is useful in a number of contexts, as further described below.

The combination may be administered directly to a subject or may be used to treat cells in culture ex vivo, which treated cells can then be administered to a subject, generally the subject from which the cells are derived (autologous transplantation) or a closely related subject (allogeneic transplantation). Each of the essential elements of the combination may be supplied as a single member of the class or may be supplied as a mixture or other combination of the members of the class. Each component of the combination (indeed, each member of the sub-combination representing a single class) can be administered independently, at the same time, by the same route, or at the same time by different routes, or at different times by the same or different routes as any other component in the combination. Thus, for example, if two different CXCR4 antagonists are used, both can be, but need not be, administered at the same time; both can be, but need not be, administered intravenously. Similarly, if two or more GROβ proteins are used, these too may be subject to the variable types of administration just described. The same applies to administration of a member of the CXCR4 antagonist class and a member of the GROβ protein class. The combination GROβ protein(s) and CXCR4 antagonist(s) may also be administered according to such variable protocols, independently or in the same composition.

The “GROβ protein” or “GROβ chemokine” class includes GROβ itself as well as modified forms of GROβ. As further described herein, these modified forms may be truncated, multimerized, contain amino acid substitutions, deletions or insertions, or may comprise combinations of these.

The CXCR4 antagonists are preferably compounds of formula (1). Other preferred CXCR4 antagonists for use in the methods of the invention are compounds of formula (1A)-(1F), (2A)-(2B), (3), (3A)-(3C), (4), (4A)-(4C), (5), (6), (6A)-(6D), (7) and (8) as shown below.

The compounds of formula (1) inhibit HIV replication via inhibition of CXCR4, the co-receptor required for fusion and entry of T-tropic HIV strains, and also inhibit the binding and signaling induced by the natural ligand, the chemokine SDF-1. While not wishing to be bound by any theory, the compounds of formula (1) which inhibit the binding of SDF-I to CXCR4 effect an increase in stem and/or progenitor cells by virtue of such inhibition. Enhancing the stem and/or progenitor cells in blood is helpful in treatments to alleviate the effects of protocols that adversely affect the bone marrow, such as those that result in leukopenia. These are known side-effects of chemotherapy and radiotherapy. The compounds of formula (1) also enhance the success of bone marrow transplantation, enhance wound healing and bum treatment, and aid in restoration of damaged organ tissue. They also combat bacterial infections that are prevalent in leukemia. As described in WO 03/011277, the compounds of formula (1) are used to mobilize and harvest CD34+ cells via apheresis with and without combinations with other mobilizing factors. The harvested cells are used in treatments requiring stem cell transplantations.

As used herein, the term “progenitor cells” refers to cells that, in response to certain stimuli, can form differentiated hematopoietic or myeloid cells. The presence of progenitor cells can be assessed by the ability of the cells in a sample to form colony-forming units of various types, including, for example, CFU-GM (colony-forming units, granulocyte-macrophage); CFU-GEMM (colony-forming units, multipotential); BFU-E (burst-forming units, erythroid); HPP-CFC (high proliferative potential colony-forming cells); or other types of differentiated colonies which can be obtained in culture using known protocols.

As used herein, “stem” cells are less differentiated forms of progenitor cells. Typically, such cells are often positive for CD34. Some stem cells do not contain this marker, however. These CD34+ cells can be assayed using fluorescence activated cell sorting (FACS) and thus their presence can be assessed in a sample using this technique.

In general, CD34+ cells are present only in low levels in the blood, but are present in large numbers in bone marrow. While other types of cells such as endothelial cells and mast cells also may exhibit this marker, CD34 is considered an index of stem cell presence.

In some compounds of formula (1), Z and Z′ are cyclic polyamine moieties having from 9-24C that include 3-5 nitrogen atoms, as described in U.S. Pat. Nos. 5,021,409; 6,001,826 and 5,583,131, incorporated herein by reference. Particularly preferred are 1,5,9,13-tetraazacyclohexadecane; 1,5,8,11,14-pentaazacyclohexadecane; 1,4,8,11-tetraazacylotetradecane; 1,5,9-triazacyclododecane; 1,4,7,10-tetraazacyclododecane; and the like, including such cyclic polyamines which are fused to an additional aromatic or heteroaromatic rings and/or containing a heteroatom other than nitrogen incorporated in the ring. These and embodiments wherein the cyclic polyamine contains a fused additional cyclic system or one or more additional heteroatoms are described in U.S. Pat. No. 5,698,546 incorporated hereinabove by reference. Also preferred are
3,7,11,17-tetraazabicyclo(13.3.1)heptadeca-1(17),13,15-triene;
4,7,10,17-tetraazabicyclo(13.3.1)heptadeca-1(17),13,15-triene;
1,4,7,10-tetraazacyclotetradecane; 1,4,7-triazacyclotetradecane; and
4,7,10-triazabicyclo(13.3.1)heptadeca-1(17),13,15-triene.

When Z′ is other than a cyclic polyamine as defined in Z, its preferred embodiments are set forth in U.S. Pat. Nos. 5,817,807; 6,756,391; 6,506,770; and 6,667,320, also incorporated herein by reference.

Forms where
Z is of the formula 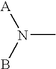
wherein A comprises a monocyclic or bicyclic fused ring system containing at least one N and B is H or an organic moiety of 1-20 atoms are disclosed in U.S. Pat. Nos. 6,734,191; 6,750,348; and application Ser. No. 09/957,682 filed 17 Sep. 2001 and now allowed; Ser. No. 09/957,654 filed 17 Sep. 2001 and now allowed, all incorporated herein by reference.

Preferred forms of the linker moiety include those wherein the linker is a bond, or wherein the linker is an alkylene or includes an aromatic moiety flanked by alkylene, preferably methylene moieties. Preferred linking groups include the methylene bracketed forms of
1,3-phenylene, 2,6-pyridine, 3,5-pyridine, 2,5-thiophene, 4,4′-(2,2′-bipyrimidine);
2,9-(1,10-phenanthroline) and the like. A particularly preferred linker is 1,4-phenylene-bis-(methylene).

In one aspect, the CXCR4 antagonist for use in the methods of the present invention may be exemplified by compounds having formula (1A):
V—CR₂—Ar¹—CR₂NR—(CR₂)ₓ—Ar² (1A)
wherein V is a substituted heterocycle of 9-24 members containing 2-4 optionally substituted amine nitrogen atoms spaced from each other by 2 or more optionally substituted carbon atoms, and which heterocycle may optionally comprise a fused aromatic or heteroaromatic ring, and wherein
(a) said heterocycle contains at least one O or S, said O or S spaced from any adjacent heteroatom by at least 2 carbon atoms, and wherein said S is optionally oxidized or
(b) at least one carbon atom in said ring is substituted by an electron-withdrawing substituent, or
(c) both (a) and (b);
and wherein each R is independently H or a straight chain, branched or cyclic alkyl containing 1-6C;
x is 0-4;
Ar¹ is an unsubstituted or substituted aromatic or heteroaromatic moiety; and
Ar² is an unsubstituted or substituted aromatic or heterocyclic group.

In the above Formula (1A), V may contain 2-4 N, preferably 3-4 N if there is no additional heteroatom. Preferable ring sizes for V are 9-18 members, more preferably 12-16 members. V may also include a fused aromatic or heteroaromatic ring, preferably 1, 2 or 1,3 or 1,4 phenylene or 2, 6 or 2, 5 or 2, 4 or 2,3 pyridinylene. The fused ring may also be, for example, 2,5 or 2,6 pyrimidinylene or 2, 4 or 2,3 pyrrolylene.

In the above Formula I, the electron withdrawing substituents present at at least one C in ring V may be halogen, nitro, cyano, carboxylic acid, a carboxylic ester formed from an alcohol of 1-6C, an amide formed from an amine of 0-12C, a sulfonic or sulfinic acid, ester or amide, CF₃, and the like. A preferred electron withdrawing substituent is ═O, as well as halo. Examples of halogen include fluorine, chlorine, bromine, iodine, with fluorine and chlorine preferred.

In the above Formula (1A), Ar² may be an optionally substituted heterocyclic group or aromatic group. Examples of aromatic groups include but are not limited to benzene, naphthalene, dihydronaphthalene and tetrahydronaphthalene. Examples of heterocyclic groups include 5 to 6-membered saturated, partially saturated, or aromatic heterocyclic rings containing 1 to 4 heteroatoms selected from nitrogen, oxygen and sulfur. The heterocycles may be pyridine, quinoline, isoquinoline, imidazole, benzimidazole, azabenzimidazole, benzotriazole, furan, benzofuran, thiazole, benzothiazole, oxazole, benzoxazole, pyrrole, indole, indoline, indazole, pyrrolidine, pyrrolidone, pyrroline, piperidine, piperazine, tetrahydroquinoline, tetrahydroisoquinoline, pyrazole, thiophene, isoxazole, isothiazole, triazole, tetrazole, oxadiazole, thiadiazole, morpholine, thiamorpholine, pyrazolidine, imidazolidine, imidazoline, tetrahydropyran, dihydropyran, benzopyran, dioxane, dithiane, tetrahydrofuran, tetrahydrothiophene, dihydrofuran, dihydrothiophene, and the like. Oxides of the nitrogen and sulfur containing heterocycles are also included in the present invention.

The optional substituents on Ar² include alkyl (1-6C), alkenyl (1-6C), alkynyl (1-6C), halo, nitro, cyano, carboxylic acid, carboxylic ester formed from an alcohol with 1-6C, an amide formed from an amine of 0-12C, a sulfonic or sulfinic acid, ester or amide, OR, SR, NR₂, OCR, OOCR, NRCOR, all wherein R is hydrogen or straight or branched chain alkyl (1-6C), an optionally substituted aromatic or heterocyclic group, CF₃, and the like. Preferred substituents include alkyl, OR, NR₂, and halo. Preferred embodiments of Ar² include phenyl, pyridinyl, pyrimidinyl and imidazolyl.

In the above Formula (1A), Ar¹ may be a 5-6 membered aromatic system which is bivalent benzene, pyridine, thiophene, pyrimidine, and the like. Ar¹ may optionally be substituted by alkyl, alkenyl, halo, nitro, cyano, CF₃, COOR, CONR₂, OCR, OOCR, NRCOR, OR, NR², SR (where R is H or alkyl 1-6C), sulfonic or sulfinic acids, esters or amides and the like. Preferred embodiments of Ar¹ are phenylene, especially 1,3 and 1,4 phenylene and pyridinylene, preferably 2,6 pyridinylene, and 3,5 pyridinylene. Preferable substituents are alkyl, OR, NR₂ and halo.

Further, in the compounds of Formula (1A), each R group may be hydrogen or alkyl of 1-2C, preferably hydrogen. The R group may be coupled to a nitrogen is hydrogen or alkyl 1-6C, preferably straight chain alkyl 1-3C, more preferably H or methyl. In one example, 1, 2, 3, 4, or 5 of the R groups are methyl or ethyl and the remaining R groups are hydrogen.

In one embodiment, the CXCR4 antagonist has formula
V—CH₂—Ar¹—CH₂NR—CH₂—Ar²
wherein V is a heterocycle as defined in formula (1A), and wherein:
(a) said heterocycle is substituted with halo or ═O; or
(b) said heterocycle contains O or S; or
(c) both (a) and (b),
and wherein Ar¹ is unsubstituted 1, 3 or 1,4-phenylene, R is H, methyl or ethyl and Ar² is unsubstituted phenyl or pyridinyl. Preferred embodiments of x are 0-2 and 1-2.

The heterocycle V may contain 3 N and at least one carbon atom in the heterocycle that is substituted by at least one fluoro substituent. The R moiety may independently be hydrogen or methyl. The number of (CR₂)ₓ groups may be 0-4, 0-2, or 1-2. The Ar¹ moiety may be 1, 3 or 1,4-phenylene. The Ar² moiety may be phenyl or pyridyl. The heterocycle V may be a 12-16 membered heterocycle, or may contain O or S as a ring member. The heterocycle V may also contain an oxidized sulfur as a ring member. In one example, at least one carbon in the heterocycle V is substituted by ═O.

Compounds of formula (1A), and methods of sythesizing such compounds are described in WO 01/44229, incorporated herein by reference. Examples of compounds of Formula (1A), its pharmaceutically acceptable salts or metal complexes thereof, include but are not limited to:
N-[4-(11-fluoro-1,4,7-triazacyclotetradecanyl)-1,4-phenylenebis(methylene)]-2-(aminomethyl)pyridine;
N-[4-(11,11-difluoro-1,4,7-triazacyclotetradecanyl)-1,4-phenylenebis(methylene)]-2-(aminomethyl)pyridine;
N-[4-(1,4,7-triazacyclotetradecan-2-onyl)-1,4-phenylenebis(methylene)]-2-(aminomethyl)pyridine;
N-[12-(5-oxa-1,9-diazacyclotetradecanyl)-1,4-phenylenebis(methylene)]-2-(aminomethyl)pyridine;
N-[4-(11-oxa-1,4,7-triazacyclotetradecanyl)-1,4-phenylenebis(methylene)]-2-(aminomethyl)pyridine;
N-[4-(11-thia-1,4,7-triazacyclotetradecanyl)-1,4-phenylenebis(methylene)]-2-(aminomethyl)pyridine;
N-[4-(11-sulfoxo-1,4,7-triazacyclotetradecanyl)-1,4-phenylenebis(methylene)]-2-(aminomethyl)pyridine;
N-[4-(11-sulfono-1,4,7-triazacyclotetradecanyl)-1,4-phenylenebis(methylene)]-2-(aminomethyl)pyridine; or
N-[4-(3-carboxo-1,4,7-triazacyclotetradecanyl)-1,4-phenylenebis(methylene)]-2-(aminomethyl)pyridine.

In another aspect, the CXCR4 compound for use in the methods of the present invention is exemplified by compounds having formula (1B):
V—CR¹R²—Ar—CR³R⁴—N(R⁵)—(CR⁶R⁷)ₓ—R⁸ (1B)
wherein V is an optionally substituted 1,4,8,11-tetraazacyclotetra-decanyl, 4,7,10,17-tetraazabicyclo[13.3.1]heptadeca-1(17),13,15-trienyl, 1,4,7-triazacyclotetra-decanyl, 4,7,10-triazabicyclo[13.3.1]heptadeca-1(17),13,15-trienyl, 1,7-diazacyclotetradecanyl, or 4,10-diazabicyclo[13.31.1]heptadeca-1(17),13,15-trienyl system;
R¹ to R⁷ may be the same or different and are independently selected from hydrogen or straight, branched or cyclic C₁₋₆ alkyl;
R⁸ is pyridyl, pyrimidinyl, pyrazinyl, imidazolyl, thiophene-yl, thiophenyl, aminobenzyl, piperidinyl, purine, piperazinyl, phenylpiperazinyl, or mercaptan;
Ar is a phenylene ring optionally substituted at single or multiple positions with alkyl, aryl, amino, alkoxy, hydroxy, halogen, carboxyl and/or carboxamido; and
x is 1 or 2.

In the above formula (1B), the V moiety may be optionally substituted by hydroxyl, alkoxy, thiol, thioalkyl, halogen, nitro, carboxy, amido, sulfonic acid, and/or phosphate.

Compounds of Formula (1B), its pharmaceutically acceptable salts or metal complexes thereof, and methods of synthesizing such compounds are described in WO 00/02870, which is incorporated herein by reference. Examples of compounds having formula (1B) include but are not limited to:
N-[1,4,8,11-tetraazacyclotetra-decanyl-1,4-phenylenebis-(methylene)]-2-(aminomethyl)pyridine;
N-[1,4,8,11-tetraazacyclotetra-decanyl-1,4-phenylenebis(methylene)]-N-methyl-2-(aminomethyl)pyridine;
N-[1,4,8,11-tetraazacyclotetra-decanyl-1,4-phenylenebis(methylene)]-4-(aminomethyl)pyridine;
N-[1,4,8,11-tetraazacyclotetra-decanyl-1,4-phenylenebis(methylene)]-3-(aminomethyl)pyridine;
N-[1,4,8,11-tetraazacyclotetra-decanyl-1,4-phenylenebis(methylene)]-(2-aminomethyl-5-methyl)pyrazine;
N-[1,4,8,11-tetraazacyclotetra-decanyl-1,4-phenylenebis(methylene)]-2-(aminoethyl) pyridine;
N-[1,4,8,11-tetraazacyclotetra-decanyl-1,4-phenylenebis(methylene)]-2-(aminomethyl)thiophene;
N-[1,4,8,11-tetraazacyclotetra-decanyl-1,4-phenylenebis(methylene)]-2-(aminomethyl)mercaptan;
N-[1,4,8,11-tetraazacyclotetra-decanyl-1,4-phenylenebis(methylene)]-2-amino benzylamine;
N-[1,4,8,11-tetraazacyclotetra-decanyl-1,4-phenylenebis(methylene)]-4-amino benzylamine;
N-[1,4,8,11-tetraazacyclotetra-decanyl-1,4-phenylenebis(methylene)]-4-(aminoethyl)imidazole;
N-[1,4,8,11-tetraazacyclotetra-decanyl-1,4-phenylenebis(methylene)]-benzylamine;
N-[4-(1,4,7-triazacyclotetra-decanyl)-1,4-phenylenebis(methylene)]-2-(aminomethyl)pyridine;
N-[7-(4,7,10,17-tetraazabicyclo[13.3.1]heptadeca-1(17),13,15-trienyl)-1,4-phenylenebis(methylene)]-2-(aminomethyl)pyridine;
N-[7-(4,7,10-triazabicyclo[13.3.1]heptadeca-1(17),13,15-trienyl)-1,4-phenylenebis(methylene)]-2-(aminomethyl)pyridine;
N-[1-(1,4,7-triazacyclotetra-decanyl)-1,4-phenylenebis(methylene)]-2-(aminomethyl)pyridine;
N-[4-[4,7,10,17-tetraazabicyclo[13.3.1]heptadeca-1(17),13,15-trienyl]-1,4-phenylenebis(methylene)]-2-(aminomethyl)pyridine;
N-[4-[4,7,10-triazabicyclo[13.3.1]heptadeca-1(17),13,15-trienyl]-1,4-phenylenebis(methylene)]-2-(aminomethyl)pyridine;
N-[1,4,8,11-tetraazacyclotetradecanyl-1,4-phenylenebis(methylene)]-purine;
1-[1,4,8,11-tetraazacyclotetradecanyl-1,4-phenylenebix(methylene)]-4-phenylpiperazine;
N-[4-(1,7-diazacyclotetradecanyl)-1,4-phenylenebis(methylene)]-2-(aminomethyl)pyridine; and
N-[7-(4,10-diazabicyclo[13.3.1]heptadeca-1(17),13,15-trienyl)-1,4-phenylenebis(methylene)]-2-(aminomethyl)pyridine.

In yet another aspect, the CXCR4 for use in the methods of the present invention may be exemplified by compounds having formula (1C):
V²—CR₉R₁₀—Ar² (1C)
wherein V² is an optionally substituted 1,4,8,11-tetraazacylcotetra-decanyl or 4,7,10,17-tetraazabicyclo[13.3.1]heptadeca-1(17),13,15-trienyl system;
R⁹ and R¹⁰ may be the same or different and are independently selected from hydrogen or straight, branched or cyclic C₁₋₆ alkyl;
Ar₂ is an aromatic or heterocyclic ring each optionally substituted at single or multiple positions with electron-donating or withdrawing groups and/or aromatic and heterocyclic groups and their alkyl derivatives thereof, and the acid addition salts and metal complexes.

In the above Formula (1C), Ar₂ may be optionally substituted with alkyl, aryl, amino, alkoxy, hydroxy, halogen, carboxyl and/or carboxamido. In particular examples, Ar₂ is optionally substituted with alkoxy, alkyl, or halogen.

Compounds having formula (1C), and methods of synthesizing the same, are described in WO 00/02870, incorporated herein by reference. Examples of compounds having formula (1C) include but are not limited to:
1-[2,6-dimethoxypyrid-4-yl(methylene)]-1,4,8,11-tetraazacyclotetradecane;
1-[2-chloropyrid-4-yl(methylene)]-1,4,8,11-tetraazacyclotetradecane;
1-[2,6-dimethylpyrid-4-yl(methylene)]-1,4,8,11-tetraazacyclotetradecane;
1-[2-methylpyrid-4-yl(methylene)]-1,4,8,11-tetraazacyclotetradecane;
1-[2,6-dichloropyrid-4-yl(methylene)]-1,4,8,11-tetraazacyclotetradecane; 1-[2-chloropyrid-5-yl(methylene)]-1,4,8,11-tetraazacyclotetradecane; and
7-[4-methylphenyl (methylene)]-4,7,10,17-tetraazabicyclo[13.3.1]heptadeca-1(17),13,15-triene.

In yet another aspect, the CXCR4 antagonist for use in the methods of the present invention may be exemplified by a compound having formula (1D):
V—R-A-R′—W (1D)
wherein V and W are independently cyclic polyamine moieties having from 9 to 32 ring members and from 3 to 8 amine nitrogens in the ring spaced by 2 or more carbon atoms from each other, and having one or more aromatic or heteroaromatic rings fused thereto,
A is an aromatic or heteroaromatic moiety when V and W have one or more aromatic or heteroaromatic moieties fused thereto, with or without an additional heteroatom other than nitrogen incorporated in the ring, or A is an aromatic or heteroaromatic moiety when V and W contain a heteroatom other than nitrogen incorporated in the ring without having one or more aromatic or heteroaromatic moieties fused thereto,
and R and R′ are each a substituted or unsubstituted alkylene chain or heteroatom-containing chain which spaces the cyclic polyamines and the moiety A.

In the above Formula (1D), R and R′ may each be methylene. In one example, A is 1,3- or 1,4-phenylene. In another example, each V and W is an unsubstituted or substituted tricyclic or bicyclic ring system containing only carbon and nitrogen atoms in the rings. One of the cyclic ring systems may be a 10 to 20 membered polyamine ring system having from 3 to 6 amine nitrogen atoms, and the ring system or systems is a fused benzyl or pyridinyl ring system.

Compounds having formula (1D), and methods of synthesizing such compounds, are described in U.S. Pat. No. 5,698,546, incorporated herein by reference. These compounds include but are not limited to:
7,7′-[1,4-phenylene-bis(methylene)]bis-3,7,11,17-tetraazabicyclo[13.3.1]heptadeca-1(17),13,15-triene;
7,7′-[1,4-phenylene-bis(methylene)]bis[15-chloro-3,7,11,17-tetraazabicyclo
[13.3.1]heptadeca-1 (17),13,15-triene];
7,7′-[1,4-phenylene-bis(methylene)]bis[15-methoxy-3,7,11,17-tetraazabicyclo[13.3.1]heptadeca-1(17),13,15-triene];
7,7′-[1,4-phenylene-bis(methylene)]bis-3,7,11,17-tetraazabicyclo[13.3.1]-heptadeca-13,16-triene-15-one;
7,7′-[1,4-phenylene-bis(methylene)]bis-4,7,10,17-tetraazabicyclo[13.3.1]-heptadeca-1(17),13,15-triene;
8,8′-[1,4-phenylene-bis(methylene)]bis-4,8,12,19-tetraazabicyclo[15.3.1]nonadeca-1(19),15,17-triene;
6,6′-[1,4-phenylene-bis(methylene)]bis-3,6,9,15-tetraazabicyclo[11.3.1]pentadeca-1 (15),11,13-triene;
6,6′-[1,3-phenylene-bis(methylene)]bis-3,6,9,15-tetraazabicyclo[11.3.1]pentadeca-1 (15),11,13-triene; and
17,17′-[1,4-phenylene-bis(methylene)]bis-3,6, 14,17,23,24-hexaazatricyclo[17.3.1.1^{8,12}]tetracosa-1(23),8,10,12(24),19,21-hexaene.

In yet another aspect, the CXCR4 antagonist for use in the methods of the present invention may be exemplified by compounds having formula (1E):
Z—R-A-R′—Y (1E)
where Z and Y are identical cyclic polyamine moieties having from 10 to 15 ring members and from 3 to 6 amine nitrogens in the ring spaced by 2 or more carbon atoms from each other, said amine nitrogens being the only ring heteroatoms,
A is an aromatic or heteroaromatic moiety other than quinoline,
R and R′ are each methylene linked to nitrogen atoms in Z and Y, the amine nitrogen atoms being otherwise unsubstituted.

In the above formula (1E), each moiety Z and Y may have 14 ring members and 4 amine nitrogens in the ring. Compounds having formula (1E), and methods of synthesizing such compounds, are described in U.S. Pat. No. 5,583,131, incorporated herein by reference. These compounds include but are not limited to:
1,1′-[1,3-phenylenebis(methylene)]-bis-1,4,8,11-tetra-azacyclotetradecane;
1,1′-[1,4-phenylenebis(methylene)]-bis-1,4,8,11-tetra-azacyclotetradecane (AMD 3100);
1,1′-[1,4-phenylene-bis-(methylene)]-bis-1,4,8,11-tetraazacyclotetradecane;
bis-zinc or bis-copper complex of 1,1′-[1,4-phenylene-bis-(methylene)]-bis-1,4,8,11-tetraazacyclotetradecane;
1,1′-[3,3′-biphenylene-bis-(methylene)]-bis-1,4,8,11-tetraazacyclotetradecane;
11,11′-[1,4-phenylene-bis-(methylene)]-bis-1,4,7,11-tetraazacyclotetradecane;
1,11′-[1,4-phenylene-bis-(methylene)]-1,4,8,11-tetraazacyclotetradecane-1, 4,7,11-tetraazacyclotetradecane;
1,1′-[2,6-pyridine-bis-(methylene)]-bis-1,4,8,11-tetraazacyclotetradecane;
1,1-[3,5-pyridine-bis-(methylene)]-bis-1,4,8,11-tetraazacyclotetradecane;
1,1′-[2,5-thiophene-bis-(methylene)]-bis-1,4,8,11-tetraazacyclotetradecane;
1,1′-[4,4′-(2,2′-bipyridine)-bis-(methylene)]-bis-1,4,8,11-tetraazacyclotetradecane;
1,1′-[2,9-(1,10-phenanthroline)-bis-(methylene)]-bis-1,4,8,11-tetraazacyclotetradecane;
1,1′-[1,3-phenylene-bis-(methylene)]-bis-1,4,7,10-tetraazacyclotetradecane;
1,1′-[1,4-phenylene-bis-(methylene)]-bis-1,4,7,10-tetraazacyclotetradecane;
1′-[5-nitro-1,3-phenylenebis(methylene)]bis-1,4,8,11-tetraazacyclotetradecane;
1′,1′-[2,4,5,6-tetrachloro-1,3-phenyleneis(methylene)]bis-1,4,8,11-tetraazacyclotetradecane;
1,1′-[2,3,5,6-tetra-fluoro-1,4-phenylenebis(methylene)]bis-1,4,8,11-tetraazacyclotetradecane;
1,1′-[1,4-naphthylene-bis-(methylene)]bis-1,4,8,11-tetraazacyclotetradecane;
1,1′-[1,3-phenylenebis-(methylene)]bis-1,5,9-triazacyclododecane;
1,1′-[1,4-phenylene-bis-(methylene)]-1,5,9-triazacyclododecane;
1,1′-[2,5-dimethyl-1,4-phenylenebis-(methylene)]-bis-1,4,8,11-tetraazacyclotetradecane;
1,1′-[2,5-dichloro-1,4-phenylenebis-(methylene)]-bis-1,4,8,11-tetraazacyclotetradecane;
1,1′-[2-bromo-1,4-phenylenebis-(methylene)]-bis-1,4,8,11-tetraazacyclotetradecane; and
1,1′-[6-phenyl-2,4-pyridinebis-(methylene)]-bis-1,4,8,11-tetraazacyclotetradecane.

In yet another aspect, the CXCR4 antagonist for use in the methods of the present invention may be exemplified by compounds having formula (1F):
Z-(A)ₙ—Y (1F)
where Z and Y are independently cyclic polyamine moieties having from 9 to 32 ring members and from 3 to 8 amine nitrogen atoms in the ring, A is a linking atom or group, and n is O or an integer from 1 to 6.

In the above formula (1F) each Z and Y moiety may have 10 to 24 ring members, or 12 to 18 ring members. Each Z and Y moiety may also have 4 to 6 amine nitrogen atoms in the ring. In one example, n is 0. In another example, A is methylene.

Compounds having formula (1F), and methods of synthesizing such compounds, are described in U.S. Pat. No. 5,021,409, incorporated herein by reference. These compounds include but are not limited to:
2,2′-bicyclam, 6,6′-bicyclam;
3,3′-(bis-1,5,9,13-tetraaza cyclohexadecane);
3,3′-(bis-1,5,8,11,14-pentaazacyclohexadecane);
methylene (or polymethylene) di-1-N-1,4,8,11-tetraaza cyclotetradecane;
3,3′-bis-1,5,9,13-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
11,11′-(1,2-ethanediyl)bis-1,4,8,11-tetraazacyclotetradecane;
11,11′-(1,2-propanediyl)bis-1,4,8,11-tetraazacyclotetradecane;
11,11′-(1,2-butanediyl)bis-1,4,8,11-tetraazacyclotetradecane;
11,11′-(1,2-pentanediyl)bis-1,4,8,11-tetraazacyclotetradecane; and
11,11′-(1,2-hexanediyl)bis-1,4,8,11-tetraazacyclotetradecane.

In another aspect, the CXCR4 antagonist for use in the methods of the present invention may be exemplified by compounds having formula (2A): 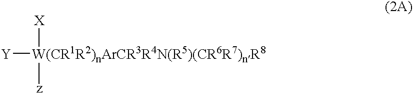
W is a nitrogen atom and Y is void, or W is a carbon atom and Y═H;
R¹ to R⁷ may be the same or different and are independently hydrogen or straight, branched or cyclic C₁₋₆ alkyl;
R⁸ is an optionally substituted heterocyclic group or an optionally substituted aromatic group
Ar is an aromatic or heteroaromatic ring optionally substituted at single or multiple, non-linking positions with electron-donating or withdrawing groups;
n and n′ are independently, 0-2;
X is a group of the formula: 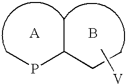
wherein, Ring A is an optionally substituted, saturated or unsaturated 5 or 6-membered ring, and P is an optionally substituted nitrogen atom and wherein any heteroatom in addition to P in ring A is N;
wherein Ring B is an optionally substituted 5 to 7-membered ring;
wherein Ring A or Ring B is bound to group W from any position through group V;
wherein V is a chemical bond or V is a (CH₂)_{n″} group (where n″=1-2), or V is a C═O group; and
wherein Z is selected from the group consisting of: a hydrogen atom; an optionally substituted C₁₋₆ alkyl group; an optionally substituted aromatic or heterocyclic group; an optionally substituted amino group; an optionally substituted C₁₋₆ alkylamino or C₃₋₇ cycloalkylamino group; and a substituted carbonyl group; or
the pharmaceutically acceptable acid addition salts thereof;
wherein said compound may be in any stereoisomeric form or present as a mixture of stereoisomeric forms thereof;
wherein Ring B is selected from the group consisting of: benzene and a 5 to 7-membered cycloalkyl ring; and the optionally substituted forms thereof.

In the above formula (2A), Ring A may be pyridine; pyrimidine; pyrazine; pyridazine; triazine; piperidine; piperazine; imidazole; pyrazole; or triazole. and the optionally substituted forms thereof. Ring B may be cyclopentyl; cyclohexyl; cycloheptyl; cyclopentenyl; cyclohexenyl; or cycloheptenyl, and the optionally substituted forms thereof. In one embodiment, Ring A and Ring B together are optionally substituted dihydroquinoline or tetrahydroquinoline.

In the above formula (2A), Ring A and Ring B are independently optionally substituted with a substituent selected from the group consisting of: halogen; nitro; cyano; carboxylic acid; an optionally substituted alkyl, alkenyl or cycloalkyl group; an optionally substituted hydroxyl group; an optionally substituted thiol group; an optionally substituted amino or acyl group; an optionally substituted carboxylate, carboxamide or sulfonamide group; and an optionally substituted aromatic or heterocyclic group. In one embodiment, the optional substituent in Ring A or Ring B is independently an optionally substituted aralkyl or heterocycloalkyl, wherein said heterocycloalkyl is a 5 or 6 membered ring containing 1-4 heteroatoms. For example, the optionally substituted aralkyl or heterocycloalkyl may be phenylC₁₋₄alkyl; phenylmethyl (benzyl); phenethyl; pyridinylmethyl; or pyridinylethyl.

In the above formula (2A), Z may be an optionally substituted C₁₋₆alkyl group, wherein said C₁₋₆alkyl group is substituted with one or more substituents selected from the group consisting of: halogen; nitro; cyano; carboxylic acid; an optionally substituted alkyl, alkenyl or cycloalkyl group; an optionally substituted hydroxyl group; an optionally substituted thiol group; an optionally substituted amino or acyl group; an optionally substituted carboxylate, carboxamide or sulfonamide group; and an optionally substituted aromatic or heterocyclic group.

In the above formula (2A), Z is an optionally substituted aromatic or heterocyclic group or a C₁₋₆alkyl group optionally substituted with an optionally substituted aromatic or heterocyclic group. In one embodiment, Z is a C₁₋₆ alkyl group substituted with an optionally substituted aromatic or heterocyclic group. The optionally substituted aromatic group may be substituted with a substituent selected from the group consisting of: benzene; naphthalene; dihydronaphthalene; and tetrahydronaphthalene; and wherein said optionally substituted heterocyclic group is a 5 to 6-membered saturated, partially saturated, or aromatic heterocyclic ring containing 1 to 4 heteroatoms selected from nitrogen, oxygen and sulfur. The heterocyclic group selected from the group consisting of: pyridine, quinoline, isoquinoline, imidazole, benzimidazole, azabenzimidazole, benzotriazole, furan, benzofuran, thiazole, benzothiazole, oxazole, benzoxazole, pyrrole, indole, indoline, indazole, pyrrolidine, pyrrolidone, pyrroline, piperidine, piperazine, tetrahydroquinoline, tetrahydroisoquinoline, pyrazole, thiophene, isoxazole, isothiazole, triazole, tetrazole, oxadiazole, thiadiazole, morpholine, thiamorpholine, pyrazolidine, imidazolidine, imidazoline, tetrahydropyran, dihydropyran, benzopyran, dioxane, dithiane, tetrahydrofuran, tetrahydrothiophene, dihydrofuran, and dihydrothiophene. The heterocyclic group may also contain nitrogen or sulfur heteroatoms; and wherein said nitrogen or sulfur heteroatoms are optionally in the form of oxides.

In another embodiment, the CXCR4 antagonist for use in the methods of the present invention may be a compound having formula (2B): 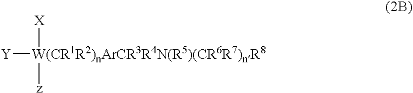
wherein, W is a nitrogen atom and Y is void;
R¹ to R⁷ may be the same or different and are independently hydrogen or straight, branched or cyclic C₁₋₆ alkyl;
R⁸ is an optionally substituted heterocyclic group or an optionally substituted aromatic group
Ar is an aromatic or heteroaromatic ring optionally substituted at single or multiple, non-linking positions with electron-donating or withdrawing groups;
n and n′ are independently, 0-2;
X is a group of the formula: 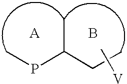
wherein, Ring A is an optionally substituted, saturated or unsaturated 5 or 6-membered ring, and P is an optionally substituted nitrogen atom and wherein any heteroatom in ring A or B is N;
wherein Ring B is an optionally substituted 5 to 7-membered ring;
wherein Ring A or Ring B is bound to group W from any position through group V;
wherein V is a chemical bond or V is a (CH₂)_{n″} group (where n″=1-2), or V is a C═O group; and
wherein Z is selected from the group consisting of: a hydrogen atom; an optionally substituted C₁₋₆ alkyl group; an optionally substituted aromatic or heterocyclic group; an optionally substituted amino group; an optionally substituted C₁₋₆ alkylamino or C₃₋₇ cycloalkylamino group; and a substituted carbonyl group; or the pharmaceutically acceptable acid addition salts thereof;
wherein said compound may be in any stereoisomeric form or present as a mixture of stereoisomeric forms thereof.

In the above formula (2B), Ring A may be pyridine; pyrimidine; pyrazine; pyridazine; triazine; piperidine; piperazine; imidazole; pyrazole; or triazole, and the optionally substituted forms thereof. Ring B may be benzene or a 5 to 7-membered cycloalkyl ring; and the optionally substituted forms thereof. For example, Ring B may be cyclopentyl; cyclohexyl; cycloheptyl; cyclopentenyl; cyclohexenyl; or cycloheptenyl. and the optionally substituted forms thereof.

In the above formula (2B), Ring A and Ring B together may be an optionally substituted dihydroquinoline or tetrahydroquinoline. For example, Ring A and Ring B are independently optionally substituted with a substituent selected from the group consisting of: halogen; nitro; cyano; carboxylic acid; an optionally substituted alkyl, alkenyl or cycloalkyl group; an optionally substituted hydroxyl group; an optionally substituted thiol group; an optionally substituted amino or acyl group; an optionally substituted carboxylate, carboxamide or sulfonamide group; and an optionally substituted aromatic or heterocyclic group. In one example, the optional substituent in Ring A or Ring B is independently an optionally substituted aralkyl or heterocycloalkyl, wherein said heterocycloalkyl is a 5 or 6 membered ring containing 1-4 heteroatoms. The optionally substituted aralkyl or heterocycloalkyl is selected from the group consisting of: phenylC₁₋₄alkyl; phenylmethyl (benzyl); phenethyl; pyridinylmethyl; and pyridinylethyl.

In the above formula (2B), Z may be an optionally substituted C₁₋₆alkyl group, wherein said C₁₋₆alkyl group is substituted with one or more substituents selected from the group consisting of: halogen; nitro; cyano; carboxylic acid; an optionally substituted alkyl, alkenyl or cycloalkyl group; an optionally substituted hydroxyl group; an optionally substituted thiol group; an optionally substituted amino or acyl group; an optionally substituted carboxylate, carboxamide or sulfonamide group; and an optionally substituted aromatic or heterocyclic group. In one example, Z is a C₁₋₆ alkyl group substituted with an optionally substituted aromatic or heterocyclic group.

In another example, Z is an optionally substituted aromatic or heterocyclic group or a C₁₋₆alkyl group optionally substituted with an optionally substituted aromatic or heterocyclic group. For example, the optionally substituted aromatic group is substituted with a substituent selected from the group consisting of: benzene; naphthalene; dihydronaphthalene; and tetrahydronaphthalene; and wherein said optionally substituted heterocyclic group is a 5 to 6-membered saturated, partially saturated, or aromatic heterocyclic ring containing 1 to 4 heteroatoms selected from nitrogen, oxygen and sulfur. The heterocyclic group may be pyridine, quinoline, isoquinoline, imidazole, benzimidazole, azabenzimidazole, benzotriazole, furan, benzofuran, thiazole, benzothiazole, oxazole, benzoxazole, pyrrole, indole, indoline, indazole, pyrrolidine, pyrrolidone, pyrroline, piperidine, piperazine, tetrahydroquinoline, tetrahydroisoquinoline, pyrazole, thiophene, isoxazole, isothiazole, triazole, tetrazole, oxadiazole, thiadiazole, morpholine, thiamorpholine, pyrazolidine, imidazolidine, imidazoline, tetrahydropyran, dihydropyran, benzopyran, dioxane, dithiane, tetrahydrofuran, tetrahydrothiophene, dihydrofuran, or dihydrothiophene. In other examples, the heterocyclic group contains nitrogen or sulfur heteroatoms; and wherein said nitrogen or sulfur heteroatoms are optionally in the form of oxides.

In one embodiment, the CXCR4 antagonist is a compound selected from the group consisting of:
N-(2-pyridinylmethyl)-N′-(6,7,8,9-tetrahydro-5H-cyclohepta[b]pyridin-9-yl)-1,4-benzenedimethanamine;
N-(2-pyridinylmethyl)-N′-(5,6,7,8-tetrahydro-8-quinolinyl)-1,4-benzenedimethanamine;
N-(2-pyridinylmethyl)-N′-(6,7-dihydro-5H-cyclopenta[b]pyridin-7-yl)-1,4-benzenedimethanamine;
N-(2-pyridinylmethyl)-N′-(1,2,3,4-tetrahydro-1-naphthalenyl)-1,4-benzenedimethanamine;
N-(2-pyridinylmethyl)-N′-(1-naphthalenyl)-1,4-benzenedimethanamine;
N-(2-pyridinylmethyl)-N′-(8-quinolinyl)-1,4-benzenedimethanamine;
N-(2-pyridinylmethyl)-N′-[2-[(2-pyridinylmethyl)amino]ethyl]-N′-(1-methyl-1,2,3,4-tetrahydro-8-quinolinyl)-1,4-benzenedimethanamine;
N-(2-pyridinylmethyl)-N′-[2-[(1H-imidazol-2-ylmethyl)amino]ethyl]-N′-(1-methyl-1,2,3,4-tetrahydro-8-quinolinyl)-1,4-benzenedimethanamine;
N-(2-pyridinylmethyl)-N′-(1,2,3,4-tetrahydro-8-quinolinyl)-1,4-benzenedimethanamine;
N-(2-pyridinylmethyl)-N′-[2-[(1H-imidazol-2-ylmethyl)amino]ethyl]-N′-(1,2,3,4-tetrahydro-1-naphthalenyl)-1,4-benzenedimethanamine;
N-(2-pyridinylmethyl)-N′-(2-phenyl-5,6,7,8-tetrahydro-8-quinolinyl)-1,4-benzenedimethanamine;
N,N′-bis(2-pyridinylmethyl)-N′-(2-phenyl-5,6,7,8-tetrahydro-8-quinolinyl)-1,4-benzenedimethanamine;
N-(2-pyridinylmethyl)-N′-(5,6,7,8-tetrahydro-5-quinolinyl)-1,4-benzenedimethanamine;
N-(2-pyridinylmethyl)-N′-(1H-imidazol-2-ylmethyl)-N′-(5,6,7,8-tetrahydro-5-quinolinyl)-1,4-benzenedimethanamine;
N-(2-pyridinylmethyl)-N′-(1H-imidazol-2-ylmethyl)-N′-(5,6,7,8-tetrahydro-8-quinolinyl)-1,4-benzenedimethanamine;
N-(2-pyridinylmethyl)-N′-[(2-amino-3-phenyl)propyl]-N′-(5,6,7,8-tetrahydro-8-quinolinyl)-1,4-benzenedimethanamine;
N-(2-pyridinylmethyl)-N′-(1H-imidazol-4-ylmethyl)-N′-(5,6,7,8-tetrahydro-8-quinolinyl)-1,4-benzenedimethanamine;
N-(2-pyridinylmethyl)-N′-(2-quinolinylmethyl)-N′-(5,6,7,8-tetrahydro-8-quinolinyl)-1,4-benzenedimethanamine;
N-(2-pyridinylmethyl)-N′-(2-(2-naphthoyl)aminoethyl)-N′-(5,6,7,8-tetrahydro-8-quinolinyl)-1,4-benzenedimethanamine;
N-(2-pyridinylmethyl)-N′-[(S)-(2-acetylamino-3-phenyl)propyl]-N′-(5,6,7,8-tetrahydro-8-quinolinyl)-1,4-benzenedimethanamine;
N-(2-pyridinylmethyl)-N′-[(S)-(2-acetylamino-3-phenyl)propyl]-N′-(5,6,7,8-tetrahydro-8-quinolinyl)-1,4-benzenedimethanamine;
N-(2-pyridinylmethyl)-N′-[3-((2-naphthalenylmethyl)amino)propyl]-N′-(5,6,7,8-tetrahydro-8-quinolinyl)-1,4-benzenedimethanamine;
N-(2-pyridinylmethyl)-N′-[2-(S)-pyrollidinylmethyl]-N′-(5,6,7,8-tetrahydro-8-quinolinyl)-1,4-benzenedimethanamine;
N-(2-pyridinylmethyl)-N′-[2-(R)-pyrollidinylmethyl]-N′-(5,6,7,8-tetrahydro-8-quinolinyl)-1,4-benzenedimethanamine;
N-(2-pyridinylmethyl)-N′-[3-pyrazolylmethyl]-N′-(5,6,7,8-tetrahydro-8-quinolinyl)-1,4-benzenedimethanamine;
N-(2-pyridinylmethyl)-N′-[2-pyrrolylmethyl]-N′-(5,6,7,8-tetrahydro-8-quinolinyl)-1,4-benzenedimethanamine;
N-(2-pyridinylmethyl)-N′-[2-thiopheneylmethyl]-N′-(5,6,7,8-tetrahydro-8-quinolinyl)-1,4-benzenedimethanamine
N-(2-pyridinylmethyl)-N′-[2-thiazolylmethyl]-N′-(5,6,7,8-tetrahydro-8-quinolinyl)-1,4-benzenedimethanamine;
N-(2-pyridinylmethyl)-N′-[2-furanylmethyl]-N′-(5,6,7,8-tetrahydro-8-quinolinyl)-1,4-benzenedimethanamine;
N-(2-pyridinylmethyl)-N′-[2-[(phenylmethyl)amino]ethyl]-N′-(5,6,7,8-tetrahydro-8-quinolinyl)-1,4-benzenedimethanamine;
N-(2-pyridinylmethyl)-N′-(2-aminoethyl)-N′-(5,6,7,8-tetrahydro-8-quinolinyl)-1,4-benzenedimethanamine;
N-(2-pyridinylmethyl)-N′-3-pyrrolidinyl-N′-(5,6,7,8-tetrahydro-8-quinolinyl)-1,4-benzenedimethanamine
N-(2-pyridinylmethyl)-N′-4-piperidinyl-N′-(5,6,7,8-tetrahydro-8-quinolinyl)-1,4-benzenedimethanamine;
N-(2-pyridinylmethyl)-N′-[2-[(phenyl)amino]ethyl]-N′-(5,6,7,8-tetrahydro-8-quinolinyl)-1,4-benzenedimethanamine;
N-(2-pyridinylmethyl)-N′-(7-methoxy-1,2,3,4-tetrahydro-2-naphthalenyl)-1,4-benzenedimethanamine;
N-(2-pyridinylmethyl)-N′-(6-methoxy-1,2,3,4-tetrahydro-2-naphthalenyl)-1,4-benzenedimethanamine;
N-(2-pyridinylmethyl)-N′-(1-methyl-1,2,3,4-tetrahydro-2-naphthalenyl)-1,4-benzenedimethanamine;
N-(2-pyridinylmethyl)-N′-(7-methoxy-3,4-dihydronaphthalenyl)-1-(aminomethyl)-4-benzamide;
N-(2-pyridinylmethyl)-N′-(6-methoxy-3,4-dihydronaphthalenyl)-1-(aminomethyl)-4-benzamide;
N-(2-pyridinylmethyl)-N′-(1H-imidazol-2-ylmethyl)-N′-(7-methoxy-1,2,3,4-tetrahydro-2-naphthalenyl)-1,4-benzenedimethanamine;
N-(2-pyridinylmethyl)-N′-(8-hydroxy-1,2,3,4-tetrahydro-2-naphthalenyl)-1,4-benzenedimethanamine;
N-(2-pyridinylmethyl)-N′-(1H-imidazol-2-ylmethyl)-N′-(8-hydroxy-1,2,3,4-tetrahydro-2-naphthalenyl)-1,4-benzenedimethanamine;
N-(2-pyridinylmethyl)-N′-(8-Fluoro-1,2,3,4-tetrahydro-2-naphthalenyl)-1,4-benzenedimethanamine;
N-(2-pyridinylmethyl)-N′-(1H-imidazol-2-ylmethyl)-N′-(8-Fluoro-1,2,3,4-tetrahydro-2-naphthalenyl)-1,4-benzenedimethanamine;
N-(2-pyridinylmethyl)-N′-(5,6,7,8-tetrahydro-7-quinolinyl)-1,4-benzenedimethanamine;
N-(2-pyridinylmethyl)-N′-(1H-imidazol-2-ylmethyl)-N′-(5,6,7,8-tetrahydro-7-quinolinyl)-1,4-benzenedimethanamine;
N-(2-pyridinylmethyl)-N′-[2-[(2-naphthalenylmethyl)amino]ethyl]-N′-(5,6,7,8-tetrahydro-8-quinolinyl)-1,4-benzenedimethanamine;
N-(2-pyridinylmethyl)-N′-[2-(isobutylamino)ethyl]-N′-(5,6,7,8-tetrahydro-8-quinolinyl)-1,4-benzenedimethanamine;
N-(2-pyridinylmethyl)-N′-[2-[(2-pyridinylmethyl)amino]ethyl]-N′-(5,6,7,8-tetrahydro-8-quinolinyl)-1,4-benzenedimethanamine;
N-(2-pyridinylmethyl)-N′-[2-[(2-furanylmethyl)amino]ethyl]-N′-(5,6,7,8-tetrahydro-8-quinolinyl)-1,4-benzenedimethanamine;
N-(2-pyridinylmethyl)-N′-(2-guanidinoethyl)-N′-(5,6,7,8-tetrahydro-8-quinolinyl)-1,4-benzenedimethanamine;
N-(2-pyridinylmethyl)-N′-[2-[bis-[(2-methoxy)phenylmethyl]amino]ethyl]-N′-(5,6,7,8-tetrahydro-8-quinolinyl)-1,4-benzenedimethanamine;
N-(2-pyridinylmethyl)-N′-[2-[(1H-imidazol-4-ylmethyl)amino]ethyl]-N′-(5,6,7,8-tetrahydro-8-quinolinyl)-1,4-benzenedimethanamine;
N-(2-pyridinylmethyl)-N′-[2-[(1H-imidazol-2-ylmethyl)amino]ethyl]-N′-(5,6,7,8-tetrahydro-8-quinolinyl)-1,4-benzenedimethanamine;
N-(2-pyridinylmethyl)-N′-[2-(phenylureido)ethyl]-N′-(5,6,7,8-tetrahydro-8-quinolinyl)-1,4-benzenedimethanamine;
N-(2-pyridinylmethyl)-N′-[[N″-(n-butyl)carboxamido]methyl]-N′-(5,6,7,8-tetrahydro-8-quinolinyl)-1,4-benzenedimethanamine;
N-(2-pyridinylmethyl)-N′-(carboxamidomethyl)-N′-(5,6,7,8-tetrahydro-8-quinolinyl)-1,4-benzenedimethanamine;
N-(2-pyridinylmethyl)-N′-[(N″-phenyl)carboxamidomethyl]-N′-(5,6,7,8-tetrahydro-8-quinolinyl)-1,4-benzenedimethanamine;
N-(2-pyridinylmethyl)-N′-(carboxymethyl)-N′-(5,6,7,8-tetrahydro-8-quinolinyl)-1,4-benzenedimethanamine;
N-(2-pyridinylmethyl)-N′-(phenylmethyl)-N′-(5,6,7,8-tetrahydro-8-quinolinyl)-1,4-benzenedimethanamine;
N-(2-pyridinylmethyl)-N′-(1H-benzimidazol-2-ylmethyl)-N′-(5,6,7,8-tetrahydro-8-quinolinyl)-1,4-benzenedimethanamine;
N-(2-pyridinylmethyl)-N′-(5,6-dimethyl-1H-benzimidazol-2-ylmethyl)-N′-(5,6,7,8-tetrahydro-8-quinolinyl)-1,4-benzenedimethanamine (hydrobromide salt);
N-(2-pyridinylmethyl)-N′-(5-nitro-1H-benzimidazol-2-ylmethyl)-N′-(5,6,7,8-tetrahydro-8-quinolinyl)-1,4-benzenedimethanamine;
N-(2-pyridinylmethyl)-N′-[(1H)-5-azabenzimidazol-2-ylmethyl]-N′-(5,6,7,8-tetrahydro-8-quinolinyl)-1,4-benzenedimethanamine;
N-(2-pyridinylmethyl)-N-(4-phenyl-1H-imidazol-2-ylmethyl)-N′-(5,6,7,8-tetrahydro-8-quinolinyl)-1,4-benzenedimethanamine;
N-(2-pyridinylmethyl)-N′-[2-(2-pyridinyl)ethyl]-N′-(5,6,7,8-tetrahydro-8-quinolinyl)-1,4-benzenedimethanamine;
N-(2-pyridinylmethyl)-N′-(2-benzoxazolyl)-N′-(5,6,7,8-tetrahydro-8-quinolinyl)-1,4-benzenedimethanamine;
N-(2-pyridinylmethyl)-N′-(trans-2-aminocyclohexyl)-N′-(5,6,7,8-tetrahydro-8-quinolinyl)-1,4-benzenedimethanamine;
N-(2-pyridinylmethyl)-N′-(2-phenylethyl)-N′-(5,6,7,8-tetrahydro-8-quinolinyl)-1,4-benzenedimethanamine;
N-(2-pyridinylmethyl)-N′-(3-phenylpropyl)-N′-(5,6,7,8-tetrahydro-8-quinolinyl)-1,4-benzenedimethanamine;
N-(2-pyridinylmethyl)-N′-(trans-2-aminocyclopentyl)-N′-(5,6,7,8-tetrahydro-8-quinolinyl)-1,4-benzenedimethanamine;
N-[[4-[[(2-pyridinylmethyl)amino]methyl]phenyl]methyl]-N-(5,6,7,8-tetrahydro-8-quinolinyl)-glycinamide;
N-[[4-[[(2-pyridinylmethyl)amino]methyl]phenyl]methyl]-N-(5,6,7,8-tetrahydro-8-quinolinyl)-(L)-alaninamide;
N-[[4-[[(2-pyridinylmethyl)amino]methyl]phenyl]methyl]-N-(5,6,7,8-tetrahydro-8-quinolinyl)-(L)-aspartamide;
N-[[4-[[(2-pyridinylmethyl)amino]methyl]phenyl]methyl]-N-(5,6,7,8-tetrahydro-8-quinolinyl)-pyrazinamide;
N-[[4-[[(2-pyridinylmethyl)amino]methyl]phenyl]methyl]-N-(5,6,7,8-tetrahydro-8-quinolinyl)-(L)-prolinamide;
N-[[4-[[(2-pyridinylmethyl)amino]methyl]phenyl]methyl]-N-(5,6,7,8-tetrahydro-8-quinolinyl)-(L)-lysinamide;
N-[[4-[[(2-pyridinylmethyl)amino]methyl]phenyl]methyl]-N-(5,6,7,8-tetrahydro-8-quinolinyl)-benzamide;
N-[[4-[[(2-pyridinylmethyl)amino]methyl]phenyl]methyl]-N-(5,6,7,8-tetrahydro-8-quinolinyl)-picolinamide;
N′-Benzyl-N-[[4-[[(2-pyridinylmethyl)amino]methyl]phenyl]methyl]-N-(5,6,7,8-tetrahydro-8-quinolinyl)-urea;
N′-phenyl-N-[[4-[[(2-pyridinylmethyl)amino]methyl]phenyl]methyl]-N-(5,6,7,8-tetrahydro-8-quinolinyl)-urea;
N-(6,7,8,9-tetrahydro-5H-cyclohepta[bacteriapyridin-9-yl)-4-[[(2-pyridinylmethyl)amino]methyl]benzamide;
N-(5,6,7,8-tetrahydro-8-quinol inyl)-4-[[(2-pyridinylmethyl)amino]methyl]benzamide;
N,N′-bis(2-pyridinylmethyl)-N′-(5,6,7,8-tetrahydro-8-quinolinyl)-1,4-benzenedimethanamine;
N,N′-bis(2-pyridinylmethyl)-N′-(6,7,8,9-tetrahydro-5H-cyclohepta[bacteriapyridin-9-yl)-1,4-benzenedimethanamine;
N,N′-bis(2-pyridinylmethyl)-N′-(6,7-dihydro-5H-cyclopenta[bacteriapyridin-7-yl)-1,4-benzenedimethanamine;
N,N′-bis(2-pyridinylmethyl)-N′-(1,2,3,4-tetrahydro-1-naphthalenyl)-1,4-benzenedimethanamine;
N,N′-bis(2-pyridinylmethyl)-N′-[(5,6,7,8-tetrahydro-8-quinolinyl)methyl]-1,4-benzenedimethanamine;
N,N′-bis(2-pyridinylmethyl)-N′[(6,7-dihydro-5H-cyclopenta[bacteriapyridin-7-yl)methyl]-1,4-benzenedimethanamine;
N-(2-pyridinylmethyl)-N-(2-methoxyethyl)-N′-(5,6,7,8-tetrahydro-8-quinolinyl)-1,4-benzenedimethanamine;
N-(2-pyridinylmethyl)-N-[2-(4-methoxyphenyl)ethyl]-N′-(5,6,7,8-tetrahydro-8-quinolinyl)-1,4-benzenedimethanamine;
N,N′-bis(2-pyridinylmethyl)-1,4-(5,6,7,8-tetrahydro-8-quinolinyl)benzenedimethanamine;
N-[(2,3-dimethoxyphenyl)methyl]-N′-(2-pyridinylmethyl)-N-(5,6,7,8-tetrahydro-8-quinolinyl)-1,4-benzenedimethanamine;
N,N′-bis(2-pyridinylmethyl)-N-[1-(N″-phenyl-N″-methylureido)-4-piperidinyl]-1,3-benzenedimethanamine;
N,N′-bis(2-pyridinylmethyl)-N-[N″-p-toluenesulfonylphenylalanyl)-4-piperidinyl]-1,3-benzenedimethanamine;
N,N′-bis(2-pyridinylmethyl)-N-[1-[3-(2-chlorophenyl)-5-methyl-isoxazol-4-oyl]-4-piperidinyl]-1,3-benzenedimethanamine;
N-[(2-hydroxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(4-cyanophenyl)methyl]-N′-(2-pyridinylmethyl)-N-(6,7,8,9-tetrahydro-5H-cyclohepta[bacteriapyridin-9-yl)-1,4-benzenedimethanamine;
N-[(4-cyanophenyl)methyl]-N′-(2-pyridinylmethyl)-N-(5,6,7,8-tetrahydro-8-quinolinyl)-1,4-benzenedimethanamine;
N-[(4-acetamidophenyl)methyl]-N′-(2-pyridinylmethyl)-N-(5,6,7,8-tetrahydro-8-quinolinyl)-1,4-benzenedimethanamine;
N-[(4-phenoxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(1-methyl-2-carboxamido)ethyl]-N,N′-bis(2-pyridinylmethyl)-1,3-benzenedimethanamine;
N-[(4-benzyloxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(thiophene-2-yl)methyl]-N′-(2-pyridinylmethyl)-N-(6,7,8,9-tetrahydro-5H-cyclohepta[bacteriapyridin-9-yl)-1,4-benzenedimethanamine;
N-[1-(benzyl)-3-pyrrolidinyl]-N,N′-bis(2-pyridinylmethyl)-1,3-benzenedimethanamine;
N-[[1-methyl-3-(pyrazol-3-yl)]propyl]-N,N′-bis(2-pyridinylmethyl)-1,3-benzenedimethanamine;
N-[1-(phenyl)ethyl]-N,N′-bis(2-pyridinylmethyl)-1,3-benzenedimethanamine;
N-[(3,4-methylenedioxyphenyl)methyl]-N′-(2-pyridinylmethyl)-N-(6,7,8,9-tetrahydro-5H-cyclohepta[b]pyridin-9-yl)-1,4-benzenedimethanamine;
N-[1-benzyl-3-carboxymethyl-4-piperidinyl]-N,N′-bis(2-pyridinylmethyl)-1,3-benzenedimethanamine;
N-[(3,4-methylenedioxyphenyl)methyl]-N′-(2-pyridinylmethyl)-N-(5,6,7,8-tetrahydro-8-quinolinyl)-1,4-benzenedimethanamine;
N-(3-pyridinylmethyl)-N′-(2-pyridinylmethyl)-N-(6,7,8,9-tetrahydro-5H-cyclohepta[b]pyridin-9-yl)-1,4-benzenedimethanamine;
N-[[1-methyl-2-(2-tolyl)carboxamido]ethyl]-N,N′-bis(2-pyridinylmethyl)-1,3-benzenedimethanamine;
N-[(1,5-dimethyl-2-phenyl-3-pyrazolinone-4-yl)methyl]-N′-(2-pyridinylmethyl)-N-(5,6,7,8-tetrahydro-8-quinolinyl)-1,4-benzenedimethanamine;
N-[(4-propoxyphenyl)methyl]-N′-(2-pyridinylmethyl)-N-(6,7,8,9-tetrahydro-5H-cyclohepta[b]pyridin-9-yl)-1,4-benzenedimethanamine;
N-(1-phenyl-3,5-dimethylpyrazolin-4-ylmethyl)-N′-(2-pyridinylmethyl)-N-(5,6,7,8-tetrahydro-8-quinolinyl)-1,4-benzenedimethanamine;
N-[H-imidazol-4-ylmethyl]-N,N′-bis(2-pyridinylmethyl)-1,3-benzenedimethanamine;
N-[(3-methoxy-4,5-methylenedioxyphenyl)methyl]-N′-(2-pyridinylmethyl)-N-(6,7,8,9-tetrahydro-5H-cyclohepta[b]pyridin-9-yl)-1,4-benzenedimethanamine;
N-[(3-cyanophenyl)methyl]-N′-(2-pyridinylmethyl)-N-(6,7,8,9-tetrahydro-5H-cyclohepta[b]pyridin-9-yl)-1,4-benzenedimethanamine;
N-[(3-cyanophenyl)methyl]-N′-(2-pyridinylmethyl)-N-(5,6,7,8-tetrahydro-8-quinolinyl)-1,4-benzenedimethanamine;
N-(5-ethylthiophene-2-ylmethyl)-N′-(2-pyridinylmethyl)-N-(6,7,8,9-tetrahydro-5H-cyclohepta[b]pyridin-9-yl)-1,4-benzenedimethanamine;
N-(5-ethylthiophene-2-ylmethyl)-N′-(2-pyridinylmethyl)-N-(5,6,7,8-tetrahydro-8-quinolinyl)-1,4-benzenedimethanamine;
N-[(2,6-difluorophenyl)methyl]-N′-(2-pyridinylmethyl)-N-(6,7,8,9-tetrahydro-5H-cyclohepta[b]pyridin-9-yl)-1,4-benzenedimethanamine;
N-[(2,6-difluorophenyl)methyl]-N′-(2-pyridinylmethyl)-N-(5,6,7,8-tetrahydro-8-quinolinyl)-1,4-benzenedimethanamine;
N-[(2-difluoromethoxyphenyl)methyl]-N′-(2-pyridinylmethyl)-N-(6,7,8,9-tetrahydro-5H-cyclohepta[b]pyridin-9-yl)-1,4-benzenedimethanamine;
N-(2-difluoromethoxyphenylmethyl)-N′-(2-pyridinylmethyl)-N-(5,6,7,8-tetrahydro-8-quinolinyl)-1,4-benzenedimethanamine;
N-(1,4-benzodioxan-6-ylmethyl)-N′-(2-pyridinylmethyl)-N-(6,7,8,9-tetrahydro-5H-cyclohepta[b]pyridin-9-yl)-1,4-benzenedimethanamine;
N,N′-bis(2-pyridinylmethyl)-N-[1-(N″-phenyl-N″-methylureido)-4-piperidinyl]-1,4-benzenedimethanamine;
N,N′-bis(2-pyridinylmethyl)-N-[N″-p-toluenesulfonylphenylalanyl)-4-piperidinyl]-1,4-benzenedimethanamine;
N-[1-(3-pyridinecarboxamido)-4-piperidinyl]-N,N′-bis(2-pyridinylmethyl)-1,4-benzenedimethanamine;
N-[1-(cyclopropylcarboxamido)-4-piperidinyl]-N,N′-bis(2-pyridinylmethyl)-1,4-benzenedimethanamine;
N-[1-(1-phenylcyclopropylcarboxamido)-4-piperidinyl]-N,N′-bis(2-pyridinylmethyl)-1,4-benzenedimethanamine;
N-(1,4-benzodioxan-6-ylmethyl)-N′-(2-pyridinylmethyl)-N-(5,6,7,8-tetrahydro-8-quinolinyl)-1,4-benzenedimethanamine;
N-[1-[3-(2-chlorophenyl)-5-methyl-isoxazol-4-carboxamido]-4-piperidinyl]-N,N′-bis(2-pyridinylmethyl)-1,4-benzenedimethanamine;
N-[1-(2-thiomethylpyridine-3-carboxamido)-4-piperidinyl]-N,N′-bis(2-pyridinylmethyl)-1,4-benzenedimethanamine;
N-[(2,4-difluorophenyl)methyl]-N′-(2-pyridinylmethyl)-N-(5,6,7,8-tetrahydro-8-quinolinyl)-1,4-benzenedimethanamine;
N-(1-methylpyrrol-2-ylmethyl)-N′-(2-pyridinylmethyl)-N-(5,6,7,8-tetrahydro-8-quinolinyl)-1,4-benzenedimethanamine;
N-[(2-hydroxyphenyl)methyl]-N′-(2-pyridinylmethyl)-N-(5,6,7,8-tetrahydro-8-quinolinyl)-1,4-benzenedimethanamine;
N-[(3-methoxy-4,5-methylenedioxyphenyl)methyl]-N′-(2-pyridinylmethyl)-N-(5,6,7,8-tetrahydro-8-quinolinyl)-1,4-benzenedimethanamine;
N-(3-pyridinylmethyl)-N′-(2-pyridinylmethyl)-N-(5,6,7,8-tetrahydro-8-quinolinyl)-1,4-benzenedimethanamine;
N-[2-(N″-morpholinomethyl)-1-cyclopentyl]-N,N′-bis(2-pyridinylmethyl)-1,4-benzenedimethanamine;
N-[(1-methyl-3-piperidinyl)propyl]-N,N′-bis(2-pyridinylmethyl)-1,4-benzenedimethanamine;
N-(1-methylbenzimidazol-2-ylmethyl)-N′-(2-pyridinylmethyl)-N-(5,6,7,8-tetrahydro-8-quinolinyl)-1,4-benzenedimethanamine;
N-[1-(benzyl)-3-pyrrol idinyl]-N,N′-bis(2-pyridinylmethyl)-1,4-benzenedimethanamine;
N-[[(1-phenyl-3-(N″-morpholino)]propyl]-N,N′-bis(2-pyridinylmethyl)-1,4-benzenedimethanamine;
N-[1-(iso-propyl)-4-piperidinyl]-N,N′-bis(2-pyridinylmethyl)-1,4-benzenedimethanamine;
N-[1-(ethoxycarbonyl)-4-piperidinyl]-N′-(2-pyridinylmethyl)-N-(5,6,7,8-tetrahydro-8-quinolinyl)-1,4-benzenedimethanamine;
N-[(1-methyl-3-pyrazolyl)propyl]-N′-(2-pyridinylmethyl)-N-(5,6,7,8-tetrahydro-8-quinolinyl)-1,4-benzenedimethanamine;
N-[1-methyl-2-(N″,N″-diethylcarboxamido)ethyl]-N,N′-bis(2-pyridinylmethyl)-1,4-benzenedimethanamine;
N-[(1-methyl-2-phenylsulfonyl)ethyl]-N′-(2-pyridinylmethyl)-N-(5,6,7,8-tetrahydro-8-quinolinyl)-1,4-benzenedimethanamine;
N-[(2-chloro-4,5-methylenedioxyphenyl)methyl]-N′-(2-pyridinylmethyl)-N-(5,6,7,8-tetrahydro-8-quinolinyl)-1,4-benzenedimethanamine;
N-[1-methyl-2-[N″-(4-chlorophenyl)carboxamido]ethyl]-N′-(2-pyridinylmethyl)-N-(5,6,7,8-tetrahydro-8-quinolinyl)-1,4-benzenedimethanamine;
N-(1-acetoxyindol-3-ylmethyl)-N′-(2-pyridinylmethyl)-N-(6,7,8,9-tetrahydro-5H-cyclohepta[b]pyridin-9-yl)-1,4-benzenedimethanamine;
N-[(3-benzyloxy-4-methoxyphenyl)methyl]-N′-(2-pyridinylmethyl)-N-(6,7,8,9-tetrahydro-5H-cyclohepta[b]pyridin-9-yl)-1,4-benzenedimethanamine;
N-(3-quinolylmethyl)-N′-(2-pyridinylmethyl)-N-(5,6,7,8-tetrahydro-8-quinolinyl)-1,4-benzenedimethanamine;
N-[(8-hydroxy)-2-quinolylmethyl]-N′-(2-pyridinylmethyl)-N-(6,7,8,9-tetrahydro-5H-cyclohepta[b]pyridin-9-yl)-1,4-benzenedimethanamine;
N-(2-quinolylmethyl)-N′-(2-pyridinylmethyl)-N-(6,7,8,9-tetrahydro-5H-cyclohepta[b]pyridin-9-yl)-1,4-benzenedimethanamine;
N-[(4-acetamidophenyl)methyl]-N′-(2-pyridinylmethyl)-N-(6, 7,8,9-tetrahydro-5H-cyclohepta[b]pyridin-9-yl)-1,4-benzenedimethanamine;
N-[1H-imidazol-2-ylmethyl]-N,N′-bis(2-pyridinylmethyl)-1,4-benzenedimethanamine;
N-(3-quinolylmethyl)-N′-(2-pyridinylmethyl)-N-(6,7,8,9-tetrahydro-5H-cyclohepta[b]pyridin-9-yl)-1,4-benzenedimethanamine;
N-(2-thiazolylmethyl)-N′-(2-pyridinylmethyl)-N-(6,7,8,9-tetrahydro-5H-cyclohepta[b]pyridin-9-yl)-1,4-benzenedimethanamine;
N-(4-pyridinylmethyl)-N′-(2-pyridinylmethyl)-N-(6,7,8,9-tetrahydro-5H-cyclohepta[b]pyridin-9-yl)-1,4-benzenedimethanamine;
N-[(5-benzyloxy)benzo[b]pyrrol-3-ylmethyl]-N,N′-bis(2-pyridinylmethyl)-1,4-benzenedimethanamine;
N-(1-methylpyrazol-2-ylmethyl)-N′-(2-pyridinylmethyl)-N-(6,7,8,9-tetrahydro-5H-cyclohepta[b]pyridin-9-yl)-1,4-benzenedimethanamine;
N-[(4-methyl)-1H-imidazol-5-ylmethyl]-N,N′-bis(2-pyridinylmethyl)-1,4-benzenedimethanamine;
N-[[(4-dimethylamino)-1-napthalenyl]methyl]-N,N′-bis(2-pyridinylmethyl)-1,4-benzenedimethanamine;
N-[1,5-dimethyl-2-phenyl-3-pyrazolinone-4-ylmethyl]-N,N′-bis(2-pyridinylmethyl)-1,4-benzenedimethanamine;
N-[1-[(1-acetyl-2-(R)-prolinyl]-4-piperidinyl]-N-[2-(2-pyridinyl)ethyl]-N′-(2-pyridinylmethyl)-1,3-benzenedimethanamine;
N-[1-[2-acetamidobenzoyl-4-piperidinyl]-4-piperidinyl]-N-[2-(2-pyridinyl)ethyl]-N′-(2-pyridinylmethyl)-1,3-benzenedimethanamine;
N-[(2-cyano-2-phenyl)ethyl]-N′-(2-pyridinylmethyl)-N-(6,7,8,9-tetrahydro-5H-cyclohepta[b]pyridin-9-yl)-1,4-benzenedimethanamine;
N-[(N″-acetyltryptophanyl)-4-piperidinyl]-N-[2-(2-pyridinyl)ethyl]-N′-(2-pyridinylmethyl)-1,3-benzenedimethanamine;
N-[(N″-benzoylvalinyl)-4-piperidinyl]-N-[2-(2-pyridinyl)ethyl]-N′-(2-pyridinylmethyl)-1,3-benzenedimethanamine;
N-[(4-dimethylaminophenyl)methyl]-N′-(2-pyridinylmethyl)-N-(6,7,8,9-tetrahydro-5H-cyclohepta[b]pyridin-9-yl)-1,4-benzenedimethanamine;
N-(4-pyridinylmethyl)-N′-(2-pyridinylmethyl)-N-(5,6,7,8-tetrahydro-8-quinolinyl)-1,4-benzenedimethanamine;
N-(1-methylbenzimadazol-2-ylmethyl)-N′-(2-pyridinylmethyl)-N-(6,7,8,9-tetrahydro-5H-cyclohepta[b]pyridin-9-yl)-1,4-benzenedimethanamine;
N-[1-butyl-4-piperidinyl]-N-[2-(2-pyridinyl)ethyl]-N′-(2-pyridinylmethyl)-1,3-benzenedimethanamine;
N-[1-benzoyl-4-piperidinyl]-N-[2-(2-pyridinyl)ethyl]-N′-(2-pyridinylmethyl)-1,3-benzenedimethanamine;
N-[1-(benzyl)-3-pyrrolidinyl]-N-[2-(2-pyridinyl)ethyl]-N′-(2-pyridinylmethyl)-1,3-benzenedimethanamine;
N-[(1-methyl)benzo[b]pyrrol-3-ylmethyl]-N-[2-(2-pyridinyl)ethyl]-N′-(2-pyridinylmethyl)-1,3-benzenedimethanamine;
N-[1H-imidazol-4-ylmethyl]-N-[2-(2-pyridinyl)ethyl]-N′-(2-pyridinylmethyl)-1,3-benzenedimethanamine;
N-[1-(benzyl)-4-piperidinyl]-N-[2-(2-pyridinyl)ethyl]-N′-(2-pyridinylmethyl)-1,4-benzenedimethanamine;
N-[1-methylbenzimidazol-2-ylmethyl]-N-[2-(2-pyridinyl)ethyl]-N′-(2-pyridinylmethyl)-1,4-benzenedimethanamine;
N-[(2-phenyl)benzo[b]pyrrol-3-ylmethyl]-N-[2-(2-pyridinyl)ethyl]-N′-(2-pyridinylmethyl)-1,4-benzenedimethanamine;
N-[(6-methylpyridin-2-yl)methyl]-N′-(2-pyridinylmethyl)-N-(5,6,7,8-tetrahydro-8-quinolinyl)-1,4-benzenedimethanamine;
N-(3-methyl-1H-pyrazol-5-ylmethyl)-N′-(2-pyridinylmethyl)-N-(5,6,7,8-tetrahydro-8-quinolinyl)-1,3-benzenedimethanamine;
N-[(2-methoxyphenyl)methyl]-N′-(2-pyridinylmethyl)-N-(5,6,7,8-tetrahydro-8-quinolinyl)-1,3-benzenedimethanamine;
N-[(2-ethoxyphenyl)methyl]-N′-(2-pyridinylmethyl)-N-(6,7,8,9-tetrahydro-5H-cyclohepta[b]pyridin-9-yl)-1,3-benzenedimethanamine;
N-(benzyloxyethyl)-N′-(2-pyridinylmethyl)-N-(5,6,7,8-tetrahydro-8-quinolinyl)-1,3-benzenedimethanamine;
N-[(2-ethoxy-1-naphthalenyl)methyl]-N′-(2-pyridinylmethyl)-N-(5,6,7,8-tetrahydro-8-quinolinyl)-1,3-benzenedimethanamine;
N-[(6-methylpyridin-2-yl)methyl]-N′-(2-pyridinylmethyl)-N-(5,6,7,8-tetrahydro-8-quinolinyl)-1,3-benzenedimethanamine;
1-[[4-[[(2-pyridinylmethyl)amino]methyl]phenyl]methyl]guanidine;
N-(2-pyridinylmethyl)-N-(8-methyl-8-azabicyclo[3.2.1]octan-3-yl)-1,4-benzenedimethanamine;
1-[[4-[[(2-pyridinylmethyl)amino]methyl]phenyl]methyl]homopiperazine;
1-[[3-[[(2-pyridinylmethyl)amino]methyl]phenyl]methyl]homopiperazine;
trans and cis-1-[[4-[[(2-pyridinylmethyl)amino]methyl]phenyl]methyl]-3,5-piperidinediamine;
N,N′-[1,4-Phenylenebis(methylene)]bis-4-(2-pyrimidyl)piperazine;
1-[[4-[[(2-pyridinylmethyl)amino]methyl]phenyl]methyl]-1-(2-pyridinyl)methylamine;
2-(2-pyridinyl)-5-[[(2-pyridinylmethyl)amino]methyl]-1,2,3,4-tetrahydroisoquinoline;
1-[[4-[[(2-pyridinylmethyl)amino]methyl]phenyl]methyl]-3,4-diaminopyrrolidine;
1-[[4-[[(2-pyridinylmethyl)amino]methyl]phenyl]methyl]-3,4-diacetylaminopyrrolidine;
8-[[4-[[(2-pyridinylmethyl)amino]methyl]phenyl]methyl]-2,5,8-triaza-3-oxabicyclo [4.3.0]nonane; and
8-[[4-[[(2-pyridinylmethyl)amino]methyl]phenyl]methyl]-2,5,8-triazabicyclo[4.3.0]nonane.

Compounds having formula (2A) and (2B) and methods for synthesizing such compounds are set forth in WO 00/56729, incorporated herein by reference.

In another aspect, the CXCR4 antagonist for use in the methods of the present invention for use in the methods of the present invention may be exemplified by compounds having formula (3): 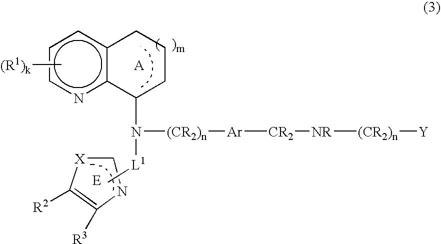
or the salts, prodrugs and stereochemical forms thereof, wherein:
Ring A optionally comprises a heteroatom selected from N, O and S;
the dotted lines represent optional unsaturation;
R¹ is halo, nitro, cyano, optionally substituted hydroxy, optionally substituted thiol, optionally substituted amino, carboxylate, carboxamide, sulfonate, sulfonamide, C2-4 alkanoyl, alkylsulfonyl, or aroyl;
R² and R³ are independently H, an optionally halogenated C1-4 alkyl, an optionally substituted aryl or heterocyclic group, or R² and R³ together with ring E may form a substituted or unsubstituted 5-7 membered ring;
k is 0-4;
m is 0-2;
L¹ is a covalent bond of C1-6 alkyl optionally containing N or O;
X is unsubstituted or substituted C, N; or O or S;
Ar is phenylene;
each n is independently 0-2;
each R is independently H or alkyl (1-6C); and
Y is a fused or unfused aromatic or heteroaromatic ring, or a 5-6 membered heterocyclic group.

In the above formula (3), Y may be a substituted or unsubstituted benzene, napthalene, dihydronapthalene, tetrahydronapthalene, pyridine, quinoline, isoquinoline, imidazole, benzimidazole, azabenzimidazole, benzotriazole, furan, benzofuran, thiazole, benzothiazole, oxazole, benzoxazole, pyrrole, indole, indoline, indazole, pyrrolidine, pyrrolidone, pyrroline, piperidine, piperazine, tetrahydroquinoline, tetrahydroisoquinoline, pyrazole, thiophene, isoxazole, isothiazole, triazole, tetrazole, oxadiazole, thiadiazole, morpholine, thiamorpholine, pyrazolidine, imidazolidine, imidazoline, tetrahydropyran, dihydropyran, benzopyran, dioxane, dithiane, tetrahydrofuran, tetrahydrothiophene, dihydrofuran, or dihydrothiophene.

In the above formula (3), L¹ may be linked to position 2 of ring E. The dotted line in ring E may further represent a double bond between the nitrogen shown and position 2. In one example, R² and R³ are connected so as to form a benzosubstituent to ring E.

In the above formula (3), ring A may be saturated. In some examples, m is 1 and k is 0 or 1.

In another embodiment, the CXCR4 antagonist for use in the methods of the present invention has formula (3A): 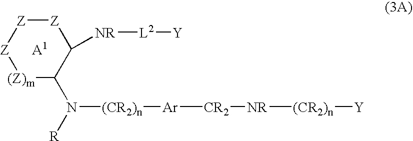
or the salts, prodrugs and stereochemical forms thereof, wherein:
R, m, n, Ar, and each Y are defined as in formula (3);
L² is a covalent bond or C1-6 alkyl optionally containing N or O;
and each Z is independently CR₂, NR, O or S, with the proviso that only two Z can be other than CR₂.

In the above formula (3A), L² may be methylene or ethylene. In one example, m is I and all Z embodiments are CR₂, particularly CH₂.

In the above formula (3A), each Y may be pyrimidyl, pyridyl, phenyl, benzimidazole or benzoxazole.

In yet another embodiment, the CXCR4 antagonist for use in the methods of the present invention has formula (3B): 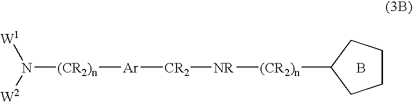
or the salts, prodrugs and stereochemical forms thereof, wherein:
W¹ is a monocyclic (5-6 membered) or fused bicyclic (8-12 membered) unsubstituted or substituted ring system containing at least one heteroatom selected from N, O and S;
W² is H, or is selected from the group consisting of: an optionally substituted C₁₋₆ alkyl group; a C₀₋₆ alkyl group substituted with an optionally substituted aromatic or heterocyclic group; an optionally substituted C₀₋₆ alkylamino or C₃₋₇ cycloalkylamino group; and an optionally substituted carbonyl group or sulfonyl;
Ar, R and n are defined as in Formula (3), and 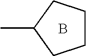
is a saturated or unsaturated 5-membered ring containing 1-2 heteroatoms selected from N, O and S.

In yet another embodiment, the CXCR4 antagonist for use in the methods of the present invention has formula (3C): 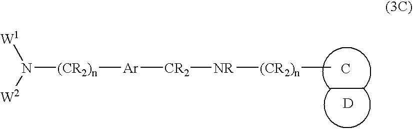
or the salts, prodrugs or stereochemical forms thereof, wherein:
W¹ is phenyl, pyridyl, pyridimyl, imidazolyl, thiophenylyl, and a fused ring system optionally having a heteroatom selected from N, O and S;
W² is H;
Ar, R and n are defined as in formula (3); and 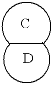
represents a fused ring system of 10 members, optionally containing 1 or 2 heteroatoms selected from N, O and S.

Compounds having formula (3), and (3A)-(3C) and methods for synthesizing such compounds are set forth in WO 02/22600, which is incorporated herein by reference.

In another aspect, the CXCR4 antagonist for use in the methods of the present invention may be exemplified by compounds having formula (4): 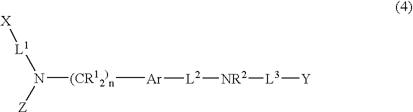
or the salts, prodrugs and stereochemical forms thereof, wherein:
X is a monocyclic (5-6 membered) or fused bicyclic (9-12 membered) unsubstituted or substituted ring system containing at least one heteroatom selected from N, O and S;
Z is H, or is an optionally substituted 5-6 membered monocyclic or 9-12 membered fused bicyclic ring system containing N, O or S;
Ar is an optionally substituted aromatic or heteroaromatic ring;
each of L¹, L² and L³ is independently a bond, CO, SO₂, or CH₂, wherein at least one of L² and L³ must comprise CO or SO₂; and wherein L¹ can also be alkylene (2-5C) wherein one or two C may optionally be replaced by N and which alkylene may itself optionally be substituted by a bridge alkylene (3-4C); L² and L³ also may be, independently, SO₂NH, CONH, SO₂NHCH₂ or CONHCH₂;
n is 0, 1 or 2;
each R¹ and R² is independently H or straight or branched chain or cyclic alkyl (1-6C) which may optionally be substituted, and wherein R² may be alkylene coupled to Y; and
Y comprises at least one aromatic or heteroaromatic or other heterocyclic substituted or unsubstituted ring coupled directly to L³.

In the above formula (4), X may be dihydroquinoline, tetrahydroquinoline, pyranopyridine, dihydropyranopyridine, thiapyranopyridine, dihydrothiapyranopyridine, dihydronaphthyridine, tetrahydronaphthyridine, imidazolyl, oxazolyl, thiazolyl, benzimidazolyl, benzothiazolyl, or benzoxazolyl.

In the above formula (4), L¹ may be alkylene (2-5C) wherein one C may optionally be replaced by N and which may optionally be substituted by a bridging alkylene (3-4C). For example, L¹ may be alkylene, CO or SO₂, and X is an optionally substituted imidazole, oxazole, thiazole, benzimidazole, benzothiazole, or benzoxazole. Alternatively, L¹ may be a bond, and X is substituted or unsubstituted dihydroquinoline, tetrahydroquinoline, pyranopyridine, dihydropyranopyridine, thiapyranopyridine, dihydrothiapyranopyridine, dihydronaphthyridine, or tetrahydronaphthyridine.

In the above formula (4), Z may be hydrogen.

In the above formula (4), Y may be an optionally substituted imidazole, benzimidazole, pyridine, pyridine, pyrimidine, or phenyl, wherein the ring nitrogen may optionally be oxidized. For example, Y may be substituted with halogen, nitrile, alkyl, —OR, —SR, —NR₂, —NRCOR, —OOCR, —COR, —CONR₂, —COOR, —NO₂, —NOH, —CF₃, where R is H or alkyl (1-6C).

In the above formula (4), each X or Z may optionally be substituted by halo, nitro, cyano, carboxy, C1-10 alkyl, C2-10 alkenyl, C3-10 cycloalkyl, hydroxy, thiol, amino, acyl, carboxylate, carbamate, carboxamide, sulfonamide, a carbonyl or sulfonyl binding to a hydrogen, or substituted with a C1-10-alkyl, C2-10 alkenyl, C3-7 cycloalkyl or a 5-6 membered monocyclic aromatic group; or X or Z may optionally be substituted by a 5-6 membered monocyclic aromatic group, naphthyl or a 5-6 membered heterocyclic ring.

In one embodiment, the compound for use in the methods of the present invention has formula (4A): 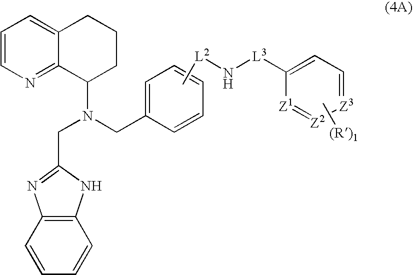
or formula (4B): 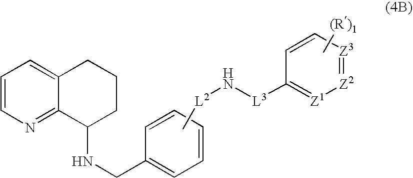
wherein 1 is 0-3, and R′ is OH, MeO, SH SMe, CN, CO₂Me, F, Cl, Br, NO₂, CH₃CO, NH₂, NHCH₃, N(CH₃)₂, CH₃CONH, CH₃SO₂NH, CONH₂, SO₂NH₂, CF₃, or Me;
each of Z¹, Z² and Z³ is independently CH, CR′ or N, wherein only two of said Z¹, Z² and Z³ can be N;
and L² and L³ are as defined in formula (4).

In the above formula (4A) or (4B), all of Z¹, Z² and Z³ may be CH or CR′. In one example, Z³ is N and L³ is CO. Furthermore, one of L² and L³ may be SO₂ and the other is a bond or CH₂. Alternatively, one of L² and L³ is CO and the other is a bond or CH₂.

In another embodiment, the compound for use in the methods of the present invention has formula (4C): 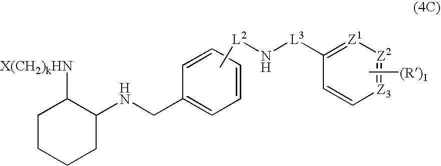
wherein 1 is 0-3, and R′ is OH, MeO, SH SMe, CN, CO₂Me, F, Cl, Br, NO₂, CH₃CO, NH₂, NHCH₃, N(CH₃)₂, CH₃CONH, CH₃SO₂NH, CONH₂, SO₂NH₂, CF₃, or Me;
k is 0-2;
each of Z¹, Z² and Z³ is independently CH, CR′ or N, wherein only two of said Z¹, Z² and Z³ can be N;
and X, L² and L³ are as defined in formula (4).

In the above formula (4C), all of Z¹, Z² and Z³ may be CH or CR′. In one example, Z³ is N and L³ is CO. Furthermore, one of L² and L³ may be SO₂ and the other is a bond or CH₂. Alternatively, one of L² and L³ may be CO and the other is a bond or CH₂.

Compounds having formula (4), and (4A)-(4C) and methods ofr synthesizing such compounds are set forth in WO 02/22599, which is incorporated herein by reference.

In another aspect, the CXCR4 antagonist for use in the methods of the present invention may be exemplified by compounds having formula (5): 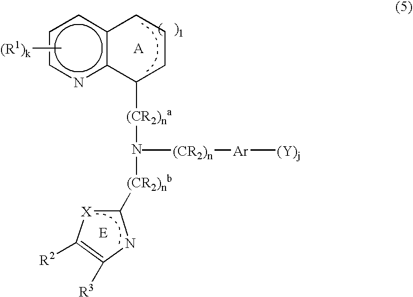
or the salts, prodrugs and stereoisomeric forms thereof;
Ring A optionally comprises a heteroatom selected from N, O and S;
the dotted lines represent optional unsaturation;
R¹, R² and R³ are independently H, halo, substituted or unsubstituted alkyl, hydroxyl, amino, thiol, or acyl; or R² and R³ may together form a benzo ring;
k is 0-4;
l is 0, 1, or 2;
X is unsubstituted or substituted C or N; or is O or S;
Ar is the residue of an aromatic or heteroaromatic moiety;
each n is independently 0-2;
each R is independently H or alkyl (1-6C);
j is 0-3; and
each Y is independently selected from the group consisting of halo, OR; SH; SO; SO₂; optionally substituted phenyl;
   —(CR₂)ₘOR;
   —(CR₂)ₘCOR;
   —(CR₂)ₘCOOR;
   —(CR₂)ₘN═CH—NR₂;
   —(CR₂)ₘCONHNHR;
   —(CR₂)ₘCN;
   —(CR₂)ₘNR⁵₂;
   —(CR₂)ₘNR(CR₂)ₘNRR⁴;
   —(CR₂)ₘNR(CR₂)ₘNR(CR₂)ₘNR⁵₂;
   —(CR₂)ₘCO(CR₂)ₘNR⁵₂;
   —(CR₂)ₘCO(CR₂)ₘNR(CR₂)ₘNRR⁴;
   —(CR₂)ₘCO(CR₂)ₘNR(CR₂)ₘNR(CR₂)ₘNR⁵₂;
   —(CR₂)ₘNRCO(CR₂)ₘNRR⁴;
   —(CR₂)ₘNRCO(CR₂)ₘNR(CR₂)ₘNR⁵₂;
   —(CR₂)ₘNRCO(CR₂)ₘNR(CR₂)ₘNR(CR₂)ₘNR(CR₂)ₘNR⁵₂;
   —(CR₂)ₘNROH;
   —(CR₂)ₘCONROH;
   —(CR₂)ₘCR═NOH;
   —NHNHR;
   —CH═N—Z; and
-guanidino or amidino, each of which may be linked to Y through a (CR₂)ₘ moiety;
wherein R is H or alkyl (1-6C), each m is independently 0-4, and each R⁴ and each R⁵ is independently H, alkyl (1-6C), alkenyl (2-6C), alkynyl (2-6C), or acyl (1-6C), each optionally substituted by one or more nonaromatic, nonheterocyclic substituent(s), wherein two R⁵ may be connected to form a cyclic amine optionally containing one or more additional heteroatoms selected from N, O and S;
a indicates the linker between Ring A and N;
b indicates the linker between ring E and the N; and
wherein Z is an aromatic or heteroaromatic moiety containing 5-12 ring members.

In the above formula (5), Ar may be a 5-6 membered monocyclic ring or a 9-12 membered fused ring system. For example, Ar may be benzene, naphthalene, dihydronaphthalene, tetrahydronaphthalene, pyridine, pyrimidine, quinoline, isoquinoline, imidazole, benzimidazole, azabenzimidazole, benzotriazole, furan, benzofuran, thiazole, benzothiazole, oxazole, benzoxazole, pyrrole, indole, imidazole, tetrahydroquinoline, tetrahydroisoquinoline, pyrazole, thiophene, isoxazole, isothiazole, triazole, tetrazole, oxadiazole, thiadiazole, imidazoline, and benzopyran. In particular exmaples, Ar is benzene, benzimidazole, benzothiazole, imidazole, oxazole, benztriazole, thiazole, pyridine, or pyrimidine. In one embodiment, at least one may be Y is —(CR₂)ₘNR⁵₂.

In the above formula (5), R² and R³ taken together may form a benzo substituent. In one embodiment, X is N and ring E comprises a pi bond coupled to one N. In one embodiment, ring E is coupled to the remainder of the molecule at position 2.

In the above formula (5), ring A may be saturated and I is 1. In one example, k is 0-1. In other examples, the ring system which includes A is tetrahydroquinoline or a substituted form thereof.

In the above formula (5), one of (CR₂)^{a}ₙ and (CR₂)^{b}ₙ may be CH₂ and the other is a bond. For example, (CR₂)^{a}ₙ may be a bond and (CR₂)^{b}ₙ is CH₂.

Compounds having formula (5) and methods for synthesizing such compounds are set forth in WO 02/34745, which is incorporated herein by reference.

In another aspect, the CXCR4 antagonist for use in the methods of the present invention may be exemplified by compounds having formula (6): 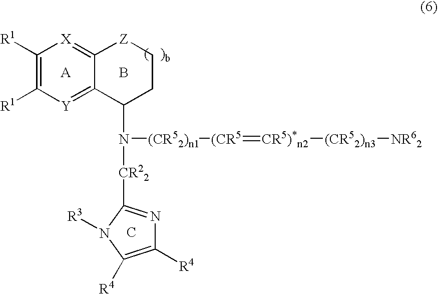
or the salts, prodrugs and stereoisomeric forms thereof,
wherein X and Y are independently N or CR¹;
Z is S, O, NR¹ or CR¹₂;
each R¹-R⁶ is independently H, halo, O(C═O)R, NR(C═O)R, OR, SR, NR², COOR, CONR₂, where R is H or optionally substituted alkyl, alkenyl, alkynyl or aryl; or
each R¹-R⁶ is alkyl (C₁₋₁₀), alkenyl (C₂₋₁₀), alkynyl (C₂₋₁₀), aryl (C₅₋₁₂), arylalkyl, arylalkenyl, or arylalkynyl, each optionally containing substituted and optionally containing O, S, or N; or an optionally substituted acyl, arylacyl, alkyl-alkenyl-, alkynyl- or arylsulfonyl wherein each alkyl, alkenyl, alkynyl or aryl moiety may contain O, O or N;
n1 is 0-4;
n2 is 0-1, wherein the * signifies C≡C may be substituted for CR⁵═CR⁵;
n3 is 0-4;
wherein n1+n2+n3 is greater than or equal to 2;
b is 0-2;
wherein the following combinations of R groups may be coupled to generate a ring, which ring may be saturated or unsaturated:
R²+R²
one R²+R³
R³+one R⁴,
R⁴+R⁴,
one R⁵+another R⁵,
one R⁵+one R⁶, and
R⁶+R⁶;
wherein the ring may not be aromatic when the participants in ring formation are two R⁵; and
wherein when n2 is 1, neither n1 nor n3 can be 0.

In one embodiment, the compounds for use in the methods of the present invention have formula (6A): 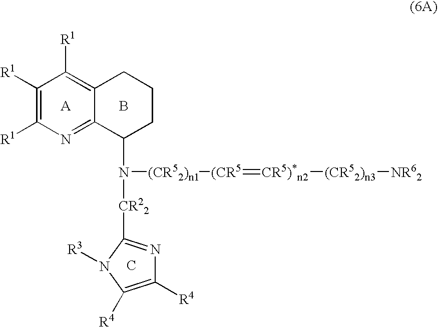
or the salts, prodrugs and stereoisomeric forms thereof,
wherein R¹-R⁶ and n1-n3 are as defined in formula (6).

In another embodiment, the compounds for use in the methods of the present invention have formula (6B) or formula (6C): 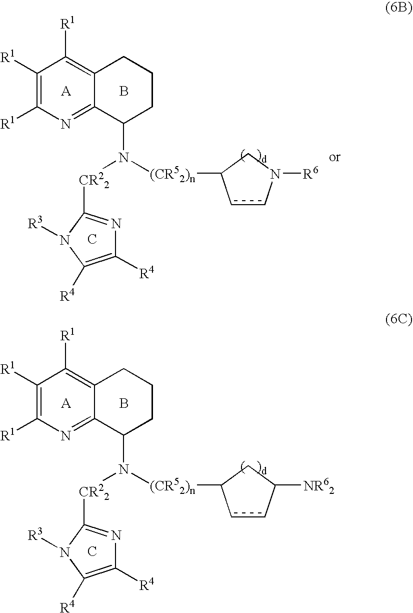
or the salts, prodrugs and stereoisomeric forms thereof,
wherein n is 0-1;
d is 0-3; the dotted line is an optional π bond; and
R¹-R⁶ are defined as in formula (6).

In yet another embodiment, the compounds for use in the methods of the present invention have formula (6D): 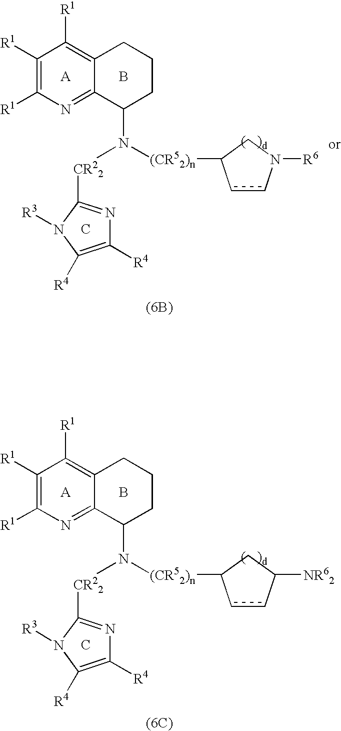
or the salts, prodrugs and stereoisomeric forms thereof,
wherein R¹-R⁶ are defined as in formula (6), and n4 is 2-6.

In the above formula (6) or (6A)-(6D), each R¹ may be H, halo, alkyl, alkoxy, or CF₃.

In one embodiment, each R² is H or alkyl. In another embodiment, each R³ is H, alkyl, alkenyl, arylalkyl, or aryl.

In the above formula (6) or (6A)-(6D), each R⁴ may be H, alkyl or aryl. Alternatively, two R⁴ may form an optionally substituted aromatic or heteroaromatic ring. For example, two R⁴ may form a phenyl or pyridyl ring, which may be substituted with halo, alkyl, halogenated alkyl, hydroxy, or alkoxy.

In the above formula (6) or (6A)-(6D), each R⁵ may be H, alkyl, or alkenyl, wherein said alkyl or alkenyl may optionally be substituted. In one embodiment, the alkyl or alkenyl substituents on a single carbon, or on nonadjacent or adjacent carbons, form a saturated or unsaturated ring. In one example, the substituents form a nonaromatic ring. In another embodiment, one R⁵ is an oxime, an alkylated oxime, alkylated hydroxylamine, hydroxylamine or halo.

In the above formula (6) or (6A)-(6D), each R⁶ may independently H, or an arylalkyl or arylsulfonyl, wherein the aryl moiety may comprise a heteroatom; or two R⁶ may comprise a guanidyl, carbonyl, or carbamino group. In one embodiment, two R⁶ together, or one R⁵ and one R⁶ together may form a saturated, unsaturated or aromatic ring, wherein each ring may optionally contain N, S or O.

Compounds having formula (6) and methods for synthesizing such compounds are set forth in WO 03/055876, which is incorporated herein by reference.

In another aspect, the CXCR4 antagonist for use in the methods of the present invention may be exemplified by compounds having formula (7): 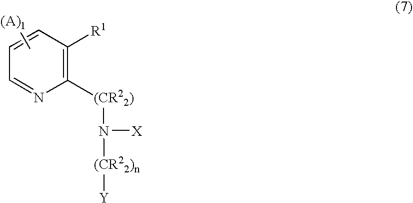
or the salts, prodrugs and stereoisomeric forms thereof,
wherein X is (CR³₂)ₒ—(CR³═CR³)ₚ—(CR³²)_{q}—NR⁵₂; (CR³₂)ᵣ—R⁴; or an optionally substituted benzyl, or a monocyclic or bicyclic ring optionally containing N, O or S;
Y is an optionally substituted 5-12 membered heterocyclic ring containing a nitrogen atom, said heterocyclic ring may be monocyclic or fused, and is aromatic or partially aromatic;
A and R¹ are independently halo, CF₃, cyano, nitro, OR, SR, NR², COOR, CONR₂, NSO₂R, OSO₂R, or OSO₂NR, where each R is H, alkyl, alkenyl, alkynyl or aryl; or A and R¹ are independently an optionally substituted alkoxy (C₁₋₁₀), alkyl (C₁₋₁₀), alkenyl (C₂₋₁₀), alkynyl (C₂₋₁₀), aryl (5-12 members), arylalkyl, arylalkenyl, or arylalkynyl, each of which may optionally contain O, S, or N;
R and R³ are independently H or an optionally substituted alkyl;
R⁴ is an optionally substituted heterocyclic ring or heteroaryl; or R⁴ comprises a urea, hydroxyurea, sulfamide, acetamide, guanidine, cyanamide, hydroxylamine, cyanamide, imidazolidine-2-one, or a nicotinamide moiety, each of which may be susbstituted with a heterocyclic ring;
R⁵ is H or alkyl;
1 and n are independently 0-4;
p is 0-1;
o and q are independently 1-4; and
r is 1-6.

In the above formula (7), at least one of R¹ and R² may not be H, and may be connected to form an additional ring such as an aryl or heteroaryl. In one example, two As may not form an additional ring. In another example, X is (CR³²)ᵣ—R⁴, r is at least two, and R⁴ is 2-pyridinyl, quinolinyl, imidazolyl or furan.

In the above formula (7), X may be (CR³₂)ₒ—(CR³═CR³)ₚ—(CR³²)_{q}—NR⁵₂, wherein each R³ and R⁵ are independently H and p may be zero. In particular embodiments, o and q together are 2-6. Alternatively, X may be (CR³₂)ᵣ—R⁴, wherein R⁴ is a heterocyclic ring or heteroaryl, each of which contains a nitrogen atom. For example, R⁴ may be azetidine, pyrrolidinyl, pyridinyl, thiophenyl, imidazolyl, or benzimidazolyl. Alternatively, X may be a monocylic or bicyclic ring optionally containing N, O or S, such as cyclohexyl, piperidine, 8-aza-bicyclo[3.2.1]octane or 3-aza-bicyclo[3.2.1]octane. In yet another embodiment, X is an optionally substituted benzyl, particularly a disubstituted benzyl.

In the above formula (7), Y may be a 5-6 membered heterocyclic ring containing a nitrogen atom adjacent to the atom that is attached to the remainder of the molecule. The 5-6 membered heterocyclic ring may be fused to another ring. For example, Y may be pyridine, pyrimidine, pyrazine, indole, benzimidazole, benzothiazole, imidazole, isoquinoline, tetrahydroquinoline, pyridazine, thiazole, or benzoimidazole. In particular examples, Y is tetrahydroquinoline, particularly a 5,6,7,8 tetrahydroquinoline moeity, attached at position 8 to the remainder of the molecule.

In the above formula (7), each optionally substituted moiety may be substituted with a heteroatom, halo, CF₃, cyano, nitro, hydroxy, alkoxy, carbonyl, carboxy, amino, amido, imino, cyano, sulfonyl; C₁₋₆ alkyl or C₂₋₆ alkenyl each of which may contain N, O, or S; or substituted with aryl, heteroaryl, carbocyclic or heterocyclic ring, each of which may further be substituted with the same substituents.

Compounds having formula (7) and methods for synthesizing such compounds are set forth in WO 04/091518, which is incorporated herein by reference.

In another aspect, the CXCR4 antagonist for use in the methods of the present invention may be exemplified by compounds having formula (8) 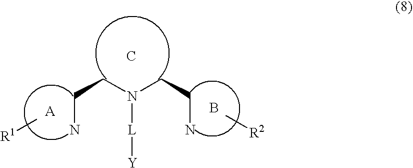
or the salts, prodrugs and stereoisomeric forms thereof,
wherein each of rings A and B is independently an optionally substituted 5-6 membered monocyclic heteroaryl;
ring C is an optionally substituted saturated or partially saturated 5-7 membered ring, and may contain a heteroatom in addition to nitrogen, wherein said heteroatom is N, O or S;
Y is H, a C₁₋₆ alkyl containing one or more heteroatoms, or a cyclic moiety, each of which is optionally substituted;
R¹ and R² are independently H, halo or an optionally substituted alkyl;
L is (CR³₂)₁ or NR(CR³₂)₁ wherein an alkyl bond may be replaced with an alkenyl or alkynyl bond;
1 is 1-6; and
each R³ is H or alkyl.

In the above formula (8), at least one of R¹ and R² may not be H when C is piperidinyl or 1,2,3,6-tetrahydropyridinyl and rings A and B are pyridinyl. In other embodiments, R¹ and R² are not both naphthalenyl when ring C is piperidinyl and rings A and B are pyridinyl. In yet other embodiments, ring C is not 4-oxo-piperidine-3,5-dicarboxylic acid if L-Y is CH₃; and ring C is not 4-hydroxy-1,2,5,6-tetrahydro-pyridine-3-carboxylic acid ester if L-Y is benzyl.

In the above formula (8), R¹ and R² may be at positions adjacent the bonds to ring C. In one example, R¹ and R² are independently unsubstituted alkyl, such as methyl.

In the above formula (8), each of rings A and B may be pyridine, pyrimidine, pyrazine, pyridazine, 1,2,3-triazine, 1,2,4-triazine, 1,3,5-triazine, 1,2,4,5-tetrazine, pyrrole, imidazole, pyrazole, 1,2,3-triazole, 1,2,4-triazole, tetrazole, thiazole, oxazole, isothiazole, isoxazole, 1,2,3-thiadiazole, 1,3,4-thiadiazole, 1,2,3-oxadiazole, 1,3,4-oxadiazole, quinoline, isoquinoline, quinoxaline, quinazoline, pthalazine, cinnoline, 1,2,3-benzotriazine, 1,2,4-benzotriazine, indole, benzimidazole, 1H-indazole, benzoxazole, benzthiazole, benz[d]isoxazole, benz[d]isothiazole, or purine. In particular examples, each of rings A and B is pyridine, pyrimidine, imidazole, or benzimidazole, and each of rings A and B may be identical. Each of rings A and B may also contain a single substituent, which may be identical, at the position adjacent to the bond linking the rings to ring C.

In the above formula (8), ring C may be a saturated ring, or may contain a double bond. For example, ring C may be pyrrolidine, piperidine, hexahydro-1H-azepine, piperazine, morpholine, thiomorpholine, azepane, azocane, 2,3,4,7-tetrahydro-1H-azepine, 2,3,6,7-tetrahydro-1H-azepine, 3-pyrroline, 1,2,3,6-tetrahydropyridine, isoindoline, 1,2,3,4-tetrahydroisoquinoline, 2,3,4,5-tetrahydro-1H-benzo[d]azepine, 2,3,4,5-tetrahydro-1H-benzo[c]azepine, cyclobutane, cyclopentane, cyclohexane, cycloheptane, cyclooctane, cyclopentene, cyclohexene, cycloheptene, cyclooctene, tetrahydropyran, tetrahydrothiopyran, oxepane, thiepane, oxocane, or thiocane. In particular examples, ring C is pyrrolidine, piperidine, piperazine or hexahydro-1H-azapine. Ring C may be substituted with an optionally substituted alkyl, halo, cyano, oxime, OR or C═N—OR, wherein R is an optionally substituted alkyl.

In the above formula (8), Y may be selected from the group consisting of:
—(CR₂)ₘNR₂,
—(CR₂)ₘNR₂(CR₃),
—(CR₂)ₘNR(CR₂)ₘNR₂,
—(CR₂)ₘNR(CR₂)ₘNR(CR₂)ₘNR₂,
—(CR₂)ₘOR,
—(CR₂)ₘCO(CR₂)ₘOR,
—(CR₂)ₘCO(CR₂)ₘNR₂,
—(CR₂)ₘCO(CR₂)ₘNR(CR₂)ₘNR₂,
—(CR₂)ₘNRCO(CR₂)ₘNR₂,
—(CR₂)ₘNR(CR₂)ₘCO₂R,
—(CR₂)ₘNR(CR₂)ₘCOR,
—(CR₂)ₘNR(CR₂)ₘSO₂R,
—(CR₂)ₘNRCO(CR₂)ₘNR(CR₂)ₘNR₂,
—(CR₂)ₘNRCO(CR₂)ₘNR(CR₂)ₘNR(CR₂)ₘNR(CR₂)ₘNR₂,
—(CR₂)ₘNR(CR₂)ₘOR,
—(CR₂)ₘ CR═NOH,
—(CR₂)ₘCONR(CR₂)ₘOR,
—(CR₂)ₘ N[(CR₂)ₘCO₂R]₂,
—(CR₂)ₘ ONRCONR₂,
—(CR₂)ₘ-Z,
—(CR₂)ₘNR—(CO)ₘZ,
—(CR₂)ₘNR —(CR₂)ₘZ, and
—(CR₂)ₘ—CR═N═Z;
wherein each R is H or an optionally substituted alkyl,
each m is independently 0-4; and
Z is an optionally substituted aromatic or heteroaromatic moiety containing 5-12 ring members.

In particular embodiments, Y is (CH₂)₁NR₂ and 1 is 1-10. Alternatively, Y may be a 5-12 membered aromatic, heteroaromatic, or a heterocyclic moiety, each of which may be a monocyclic or fused ring. For example, Y may be phenyl, imidazole, pyridine, thiophene, pyrrolidine, pyrazole, piperidine, azetidine, benzimidazole, benzo[d]isoxazole, or thiazole. Furthermore, Y may optionally be substituted with halo; cyano; nitro; alkoxy; halogenated alkyl; substituted carbonyl; a cyclic moiety such as a 5-12 membered aryl or heteroaryl containing N, O or S; or an alkyl, alkenyl, or a heteroalkyl moiety optionally containing one or more N, O, S, each of which is optionally substituted and optionally in the form of oxides. In particular examples, Y is substituted with pyridine, phenyl, piperidine or 2H-tetrazole.

In the above formula (8), each optionally substituted group may be substituted with inorganic moieties such as a heteroatom, halo, nitro, hydroxy, carboxy, amino, amido, cyano, or sulfonyl; or may be substituted with alkyl (C₁₋₁₀), alkenyl (C₂₋₁₀), alkynyl (C₂₋₁₀), aryl (5-12 members), arylalkyl, arylalkenyl, and arylalkynyl, each of which may optionally contain a heteroatom such as O, S, or N, and each of which may further be substituted with the same substituents. For example, each optionally substituted alkyl may be substituted with a heteroatom such as N, O, or S, or with a carbocyclic, heterocyclic, aryl or heteroaryl substituent.

Compounds having formula (8) and methods for synthesizing such compounds are set forth in WO 04/093817, and in U.S. patent application Ser. No. 10/977,221, filed 28 Oct. 2004, each of which is incorporated herein by reference.

Other CXCR4 inhibitors that may be used to practice the methods of the invention include but are not limited to CTCF-0214; CTCF-9908; CP-1221 (linear peptides, cyclic peptides, natural amino-acids, unnatural amino acids, and peptidomimetic compounds); T140 and analogs; 4F-benzoyl-TN24003; KRH-1120; KRH-1636; KRH-2731; polyphemusin analogue; ALX40-4C; or those described in WO 01/85196; WO 99/50461; WO 01/94420; WO 03/090512, each of which is incorporated by reference herein.

Other agents that may be used either as single agents or in combination with CXCR4 inhibitors above, include the following: cyclophosphamide; gemcitabine; cyclosporin; Rituxan; Thalidomide; Clofarabine; Velcade; Antegren; Ontak; Revlimid (Thalidomide analog); Prochymal; Genasense/Oblimersen; Gleevec; Glivec (imatinib); Tamibarotene; Nelarabine; galium nitrate; PT-100; Bexxar; Zevalin; Pixantrone; Onco-TCS; agents that are topoisomerate inhibitor; recombinant G-CSF (filgrastim; lenograstim; ETRX101; and TLK199/Telintra); recombinant GM-CSF (sargramostim, molgramostim); recombinant SCF (ancestim); covalent conjugate of recombinant G-CSF (peffilgrastim) and the like.

Particularly preferred embodiments of the compound of the formula (1) include 2,2′-bicyclam; 6,6′-bicyclam; the embodiments set forth in U.S. Pat. Nos. 5,021,409, and 6,001,826, and in particular 1,1′-[1,4-phenylene-bis(methylene)]-bis-1,4,8,11-tetraazacyclotetradecane, set forth in U.S. Pat. No. 5,583,131, and designated herein AMD3100. Also preferred are N′-(1H-benzimidazol-2-yl methyl)-N′-(5,6,7,8-tetrahydroquinoline-8-yl)-butane-1,4-diamine as described in WO 03/055876. Other methods to synthesize the compounds useful in the method of the invention are set forth in the U.S. patents and applications above as well as U.S. Pat. No. 6,489,472 and provisional application 60/553,589 filed 15 Mar. 2004.

The compounds of the invention may be prepared in the form of prodrugs, i.e., protected forms which release the compounds of the invention after administration to the subject. Typically, the protecting groups are hydrolyzed in body fluids such as in the bloodstream thus releasing the active compound or are oxidized or reduced in vivo to release the active compound. A discussion of prodrugs is found in Smith and Williams Introduction to the Principles of Drug Design, Smith, H. J.; Wright, 2^{nd} ed., London (1988).

The compounds of formula (1), as they are amines, may be administered prepared in the forms of their acid addition salts or metal complexes thereof. Suitable acid addition salts include salts of inorganic acids that are biocompatible, including HCl, HBr, sulfuric, phosphoric and the like, as well as organic acids such as acetic, propionic, butyric and the like, as well as acids containing more than one carboxyl group, such as oxalic, glutaric, adipic and the like. Typically, at physiological pH, the compounds of the invention will be in the forms of the acid addition salts. Particularly preferred are the hydrochlorides. In addition, when prepared as purified forms, the compounds may also be crystallized as the hydrates. Those forms of the compounds of formula (1) which contain chiral centers may be optically pure or may contain a mixture of stereoisomers, including racemic mixtures or mixtures of varying optical purity.

As stated above, the compounds of formula (1) are employed in combination with GROβ, including modified forms thereof. “Modified forms of GROβ” includes truncated forms thereof, such as those described in U.S. Pat. Nos. 6,447,766; 6,399,053; 6,080,398; PCT publication 99/26645; PCT publication WO 97/15595; PCT publication WO 02/02132; PCT publication WO 97/15594; and PCT publication WO 94/29341. Also included in “modified forms of GROβ” are multimeric forms thereof. Thus “modified forms” include those with truncation of between 2 to about 8 amino acids at the amino terminus of the mature protein, truncation of between about 2 to about 10 amino acids at the carboxy terminus of the mature protein, multimeric forms of the modified and/or truncated proteins, e.g., dimers, trimers, tetramers and other aggregated forms. Particularly preferred are truncated forms of GROβ and in particular SB251353 which consists of amino acids 5-73 and forms thereof where amino acid 69 is deamidated.

The CXCR4 inhibitors including mixtures thereof are administered in combination with the chemokine GROβ and/or its modified forms. Additional active ingredients that are therapeutically or nutritionally useful may also be employed, such as antibiotics, vitamins, herbal extracts, anti-inflammatories, glucose, antipyretics, analgesics, cyclosphoshamide, recombinant G-CSF (Neupogen, Granocyte/Neutrogin, and Stemgen), and covalent conjugate of recombinant G-CSF (Neulasta) granulocyte-macrophage colony stimulating factor (GM-CSF) (such as Leukine, and Luecomax), ETRX-101, TLK 199/TILENTRA™, CTCE-0214 (Truncated SDF-1 alpha peptide analog of SFD-1), VLA-4 inhibitors, Interleukin-1 (IL-1), Interleukin-3 (IL-3), Interleukin-8 (IL-8), PIXY-321 (GM-CSF/IL-3 fusion protein), macrophage inflammatory protein, stem cell factor, thrombopoietin, other members of the GRO family or chemotherapy and the like. These may all be used together with stem cell expansion systems or kits that are medical devices, such as Replicell, Allogen, and TransStem Device TransCord Device ACE System.

Formulations for administration to animal subject use commonly understood formulation techniques well known in the art. Formulations which are suitable for particular modes of administration and for compounds of the type represented by those of formula (1) may be found in Remington's Pharmaceutical Sciences, latest edition, Mack Publishing Company, Easton, Pa.; similarly, methods for administering polypeptides such as those represented by GROβ and the modified forms thereof are found in this source.

Preferably, the compounds are administered by injection, most preferably by intravenous injection, but also by subcutaneous or intraperitoneal injection, and the like. Additional parenteral routes of administration include intramuscular and intraarticular injection. For intravenous or parenteral administration, the compounds are formulated in suitable liquid form with excipients as required. The compositions may contain liposomes or other suitable carriers. For injection intravenously, the solution is made isotonic using standard preparations such as Hank's solution.

Besides injection, other routes of administration may also be used. The compounds may be formulated into tablets, capsules, syrups, powders, or other suitable forms for administration orally. By using suitable excipients, these compounds may also be administered through the mucosa using suppositories or intranasal sprays. Transdermal administration can also be effected by using suitable penetrants and controlling the rate of release.

The formulation and route of administration chosen will be tailored to the individual subject, the nature of the condition to be treated in the subject, and generally, the judgment of the attending practitioner.

Suitable dosage ranges for the CXCR4 inhibitor vary according to these considerations, but in general, the compounds when administered alone are administered in the range of about 0.1 μg/kg-5 mg/kg of body weight; preferably the range is about 1 μg/kg-300 μg/kg of body weight; more preferably about 10 μg/kg-100 μg/kg of body weight. For a typical 70-kg human subject, thus, the dosage range is from about 0.7 μg-350 mg; preferably about 700 μg-21 mg; most preferably about 700 μg-7 mg. As the methods of the invention involve a combination of at least one compound of formula (1) with a GROβ related chemokine, lower dosages, typically 2× lower, more typically 4× lower are advantageously employed. The combination of at least one CXCR4 inhibitor and the GROβ composition may be administered together in a single formulation, simultaneously in separate formulations by the same or different routes, or at staggered times, again by the same or different routes. Optimization of the protocols for administration to a particular subject is well within ordinary skill.

The CXCR4 inhibitor and the GROβ composition chemokines may be administered as a single bolus dose, a dose over time, as in i.v. or transdermal administration, or in multiple dosages.

In addition to direct administration to the subject, the combinations of the invention can be used in ex vivo treatment protocols to prepare cell cultures which are then used to replenish the blood cells of the subject. Ex vivo treatment can be conducted on autologous cells harvested from the peripheral blood or bone marrow or from allografts from matched donors. The concentration of the compound or compounds that inhibit CXCR4 combination with the GROβ composition and optionally other agents, is a matter of routine optimization.

Subjects that will respond favorably to the method of the invention include medical and veterinary subjects generally, including human patients. Among other subjects for whom the methods of the invention is useful are cats, dogs, large animals, avians such as chickens, and the like. In general, any subject who would benefit from an elevation of progenitor cells and/or stem cells, or whose progenitor cells and/or stem cells are desirable for stem cell transplantation are appropriate for the invention method.

Typical conditions which may be ameliorated or otherwise benefited by the method of the invention include hematopoietic disorders, such as aplastic anemia, leukemias, drug-induced anemias, and hematopoietic deficits from chemotherapy or radiation therapy, including neutropenia, and thrombocytopenia. The method of the invention is also useful in enhancing the success of transplantation during and following immunosuppressive treatments as well as in effecting more efficient wound healing and treatment of bacterial inflammation. The method of the present invention is further useful for treating subjects who are immunocompromised or whose immune system is otherwise impaired. Typical conditions which are ameliorated or otherwise benefited by the method of the present invention, include those subjects who are infected with a retrovirus and more specifically who are infected with human immunodeficiency virus (HIV). The method of the invention thus targets a broad spectrum of conditions for which elevation of progenitor cells and/or stem cells in a subject would be beneficial or, where harvesting of progenitor cells and/or stem cell for subsequent stem cell transplantation would be beneficial. The combinations of the invention are also administered to regenerate myocardium by mobilizing bone marrow stem cells.

Having now generally described the invention, the same will be more readily understood through reference to the following example, which is provided by way of illustration, and are not intended to be limiting of the present invention, unless specified.

### EXAMPLE 1

### Preparation of 1,1′-[1,4-phenylenebis (methylene)]bis 1,4,8,11-tetraazacyclotetradecane

1,1′-[1,4-phenylenebis (methylene)]-bis-tris-(trifluoroacetyl)-1,4,8,11-azatetradecane (3.30 g, 3.05 mmol) was dissolved in MeOH (6.0 mL). K.sub.2 CO.sub.3 (1.27 g, 9.1 mmol) was added in one portion. The suspension was heated at reflux for 3 h. Toluene (30 mL) was then added to the cooled mixture. MeOH was removed by forming an azeotrope with toluene. After all MeOH was removed, the hot toluene solution suspended with inorganic salt was filtered and concentrated to give AMD3100 free base (1.32 g, 86%) as a white solid. All characteristics of this product are in good agreement with an authentic sample prepared according to reported methods.

### EXAMPLE 2

### Preparation of N′-(1H-benzimidazol-2-ylmethyl)-N′-(5,6,7,8-tetrahydroquinoline-8-yl)-butane-1,4-diamine

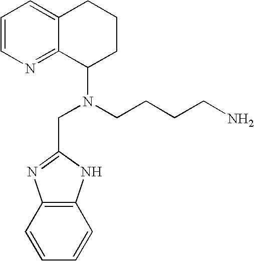

To a solution of (1-tert-butoxycarbonyl-1H-Benzimidazol-2-ylmethyl)-(5,6,7,8-tetrahydro-quinolin-8-yl)-amine (0.169 g, 0.451 mmol) in CH₃CN (5 mL) was added N,N-diisopropylethylamine (0.25 mL, 1.44 mmol) followed by 4-bromobutyronitrile (0.10 mL, 1.01 mmol). The resultant mixture was heated to 80° C. for 5 d then cooled to room temperature. The mixture was concentrated and the residue was partitioned between CH₂Cl₂ (20 mL) and brine (10 mL). The phases were separated and the aqueous phase was extracted with CH₂Cl₂ (3×10 mL). The combined organic extracts were dried (Na₂SO₄) and concentrated. Purification of the crude material by column chromatography on silica gel (30: 1:1 CH₂Cl₂-CH₃OH—NH₄OH) provided 108 mg (54%) of a yellow foam.

The intermediate from above (108 mg, 0.24 mmol) was dissolved in NH₃ saturated methanol (4 mL), treated with Raney nickel (100 mg), and placed under 50 psi H₂ on a Parr shaker, for 24 h. The mixture was filtered through Celite® and the cake was washed with methanol. The eluant was concentrated under reduced pressure. Purification of the crude material by radial chromatography on silica gel (1 mm plate, 20:1:1 CH₂Cl₂—CH₃OH—NH₄OH) provided 33 mg (39%) of the free base of the title compound as a white foam.

Conversion of the white foam (33 mg) to the hydrobromide salt, followed by re-precipitation of the intermediate solid from methanol/ether, gave the desired compound (40 mg) as a white solid. ¹H NMR (D₂O) δ 1.52 (br s, 4H), 1.74-1.88 (m, 1H), 1.95-2.08 (m, 1H), 2.15-2.21 (m, 1H), 2.34-2.39 (m, 1H), 2.50-2.61 (m, 1H), 2.79-2.86 (m, 3H), 2.99-3.02 (m, 2H), 4.38 (d, 1H, J=16.8 Hz), 4.47-4.56 (m, 2H), 7.58-7.63 (m, 2H), 7.76-7.88 (m, 3H), 8.34 (d, 1H, J=7.8 Hz), 8.62 (d, 1H, J=5.7 Hz); ¹³C NMR (D₂O) δ 20.42(2 carbons), 25.03, 25.42, 27.64, 39.50, 48.20, 51.71, 60.64, 114.26, 125.93, 126.93, 131.05, 139.32, 140.62, 148.09, 150.31, 151.82; ES-MS m/z 350 (M+H). Anal. Calcd. for C₂₁H₂₇N₅.2.9HBr.2.2H₂O: C, 40.44; H, 5.54; N, 11.23; Br, 37.15. Found: C, 40.38; H, 5.42; N, 10.85; Br, 37.42.

### EXAMPLE 3

### Preparation of N′-(1H-benzimidazol-2-ylmethyl)-N′-(S)-5,6,7,8-tetrahydro-quinolin-8-yl-butane-1,4-diamine (hydrochloride salt)

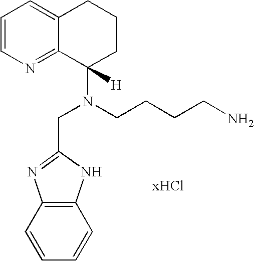

### Preparation of 4-phthalamido-butyraldehyde

A solution of 4-amino-1-butanol (5.0 g, 56 mmol) and phthalic anhydride (8.3 g, 56 mmol) in 20% MeOH/CHCl₃ (140 mL) was stirred at reflux for 66 h. The mixture was cooled to room temperature and washed sequentially with water (3×75 mL) and 1N NaOH (3×50 mL). The separated organic layer was dried (MgSO₄), concentrated, and purified by flash chromatography (5 cm id., 120 g silica gel, eluted with 2% MeOH/CH₂Cl₂) to give the desired alcohol as a white solid (4.21 g, 34%).

To a stirred slurry of TPAP (340 mg, 0.96 mmol), NMO (3.4 g, 29 mmol) and 3A molecular seives (10 g) in CH₂Cl₂ (100 mL) was added dropwise a solution of the alcohol from above (4.2 g, 19 mmol) in CH₂Cl₂ (50 mL) over 30 min. The black slurry was stirred under N₂ for 30 min after the addition, concentrated in vacuo, and purified by flash chromatography (5 cm id., 80 g silica gel, eluted with EtOAc) to afford the pure title compound as a grey solid (3.30 g, 80%). ¹H NMR (CDCl₃) δ 1.97-2.07 (m, 2H), 2.54 (t, 2H, J=7.2 Hz), 3.74 (t, 2H, J=6.8 Hz), 7.71-7.75 (m, 2H), 7.82-7.88 (m, 2H), 9.77 (s, 1H).

Using General Procedure B: 4-phthalamido-butyraldehyde from above (3.21 g, 14.8 mmol) was reacted with S-(5,6,7,8-tetrahydro-quinolin-8-yl)-amine (2.40 g, 16.3 mmol) and NaBH(OAc)₃ (9.54 g, 45.0 mmol) in dichloromethane (150 mL). Flash chromatography (5 cm id, 200 g silica gel, eluted with 5% MeOH/CH₂Cl₂) provided the pure 2° amine as a white foamy solid (2.48 g, 48%).

To a solution of the amine from above (2.5 g, 7.1 mmol) in acetonitrile (70 mL) was added diisopropylethylamine (1.9 mL, 10.7 mmol), 1-boc-2-chloromethylbenzimidazole (2.3 g, 8.6 mmol), and potassium iodide (115 mg, 0.70 mmol). The mixture was stirred under an N₂ atmosphere at 60° C. for 15 h, cooled to room temperature and concentrated in vacuo. The residue was partitioned between chloroform (150 mL) and water (100 mL). The separated organic layer was dried (MgSO₄), concentrated, and purified by flash chromatography (5 cm id, 120 g silica gel, eluted with CH₂Cl₂ to remove unreacted chloride then 2% MeOH/CH₂Cl₂ to remove desired product) to give the desired amine as a pale yellow foamy solid (3.50 g, 85%).

A solution of the amine from above (3.33 g, 5.7 mmol) in ethanol (30 mL) was treated with hydrazine monohydrate (1.80 g, 36 mmol), stirred for three hours. The mixture was then concentrated in vacuo and purified by flash chromatography (5 cm id., 80 g silica gel, eluted with 5% MeOH/CH₂Cl₂) to give the unprotected amine as a pale yellow foamy solid (1.70 g, 86%).

The amine from above (1.70 g, 4.86 mmol) was dissolved in glacial acetic acid (5 mL) and treated with HCl saturated acetic acid (5 mL). The solution was allowed to stir at room temperature 5 min, then it was slowly dropped into diethyl ether (400 mL) with vigorous stirring. The resultant slurry was suction filtered through a glass fritted funnel and the filter cake was washed with diethyl ether (3×100 mL) and dried in a vacuum oven at 40° C. for 16 h to give the desired compound as a white solid (2.34 g, 94%). ¹H NMR (D₂O) δ 1.46-1.63 (m, 4H), 1.70-1.87 (m, 1H), 1.97-2.07 (m, 1H), 2.10-2.21 (m, 1H), 2.28-2.38 (m, 1H), 2.55-2.65 (m, 1H), 2.81-2.90 (m, 3H), 2.91-3.00 (m, 2H), 4.30 (d, 1H, J=16.3 Hz), 4.41 (d, 1H, J=16.3 Hz), 4.42-4.48 (m, 1H), 7.48-7.51 (m, 2H), 7.70-7.75 (m, 3H), 8.20 (d, 1H, J=8.2 Hz), 8.53 (d, 1H, J=4.5 Hz); ¹³C NMR (D₂O) δ 20.36, 20.43, 21.67, 24.99, 25.24, 27.60, 39.51, 48.29, 51.78, 60.54, 114.46(2 carbons), 125.63, 126.10(2 carbons), 132.53, 139.58, 140.16, 147.34, 151.41, 151.81. ES-MS m/z 350 (M+H). Anal. Calcd. for C₂₁H₂₇N₅.2.5HCl.2.0H₂O.0.6CH₃COOH: C, 52.01; H, 7.06; N, 13.66; Cl, 17.29. Found: C, 52.15; H, 7.09; N, 13.40; Cl, 17.56.

The enantiomeric purity of the compound was determined to be 96.7% by chiral HPLC using the following conditions: Instrument: Hewlett Packard 1100 HPLC (VWDI); Column: Chiralpak OD, 0.46 cm×25 cm; Mobile Phases: A: 90:10 hexanes/isopropanol with 0.1% DEA, B: isopropanol; Isocratic: 90% A, 10% B; Total Run Time: 20 min; Flow Rate: 0.5 mL/min; Temperature: 10° C.; Detector: UV @ 270 nm; Injection volume: 20 μL.

Retention time of the S enantiomer=16.3 min. Retention time of the R enantiomer=21.9 min.

### EXAMPLE 4

Experimental procedures are as previously described (Pelus, L. M., et al., Blood (2001) 97:1534-1542; Blood (2004) 103:110-119). AMD3100 is 1,1′-[1,4-phenylene-bis(methylene)]-bis-1,4,8,11-tetraazacyclotetradecane.

Peripheral Blood Mobilization Using a Combination of AMD3100 and GROβ Compared to AMD3100 Alone or GROβ Alone.

Mobilization experiments were performed in BALB/c mice (3 mice group). For mobilization with AMD3100 or GROβ alone, PBSC mobilization was quantified at the peak of mobilization determined for either agent in previous experiments. Thus, PBSC mobilization was quantified at 15 mins following an s.c. injection of 2.5 mg/kg GROβ and at 1 hour following an s.c. injection of 5 mg/kg AMD3100.

In the combination experiment, PBSC mobilization was quantitated at 5, 15, 30, 60 and 150 minutes after a single subcutaneous injection of 2.5 mg/kg GROβ (R & D Systems) in saline and 5 mg/kg AMD3100. The compounds were injected separately and simultaneously at different s.c. sites. Injections were scheduled so that control and mobilized mice were evaluated at the same time in every experiment. Mice were killed by CO₂ asphyxiation and blood was obtained by cardiac puncture using syringes coated with EDTA (ethylenediaminetetra acetic acid). PBMC's were obtained by separation of peripheral blood (0.4 mL) on Lympholyte-M (Cedarlane Labs, Hornby, ON, Canada). Complete blood counts (CBC's) were performed on a Hemavet Mascot (CDC Technologies, Oxford, Conn.). Manual differentials were performed on Wright-Giemsa-stained (Hema-Tek 1000, Bayer, Elkhart, Ind.) blood smears or spleen and bone marrow cell cytospin preparations (Shandon, Pittsburgh, Pa.).

CFU-GM Assay

PBMC's were assayed for CFU-GM in McCoy 5A media with 15% heat-inactivated fetal bovine serum (Hyclone Sterile Systems, Logan, Utah) and 0.3% agar (Difco Laboratories, Detroit, Mich.). PBMC's were cultured at 2×10⁵/mL. CFU-GM were stimulated with 10 ng/mL recombinant murine GM-CSF (rmGM-CSF), 10 ng/mL rmIL-1α, and 50 ng/mL stem cell factor (SCF). Triplicate cultures from individual animals were incubated at 37° C., 5% CO₂, 5% O₂ in air for 7 days. Total CFU-GM/mL blood was determined by multiplying CFU frequencies by PBMC/ml blood corrected for white blood cell (WBC) recovery after Lympholyte-M separation.

TABLE 1

| | | MOBILIZATION | | CFU-GM |
|---|---|---|---|---|
| | | SUMMARY | | Mobilized |
| | | CFU-GM/ml | Fold Inc | Above Control |
| PBS | X | 122.04 | | |
| | SEM | 24.9 | | |
| GROβ 15 min | X | 2505.54 | 20.53 | 2383.50 |
| | SEM | 929.5 | 7.62 | 929.5 |
| AMD3100 60 min | X | 698.19 | 5.72 | 576.15 |
| | SEM | 148.7 | 1.22 | 148.7 |
| AMD3100 & | X | 3616.13 | 29.63 | 3494.09 |
| GROβ 5 min | SEM | 599.3 | 4.91 | 599.3 |
| AMD3100 & | X | 8695.44 | 71.25 | 8573.40 |
| GROβ 15 min | SEM | 648.4 | 5.31 | 648.4 |
| AMD3100 & | X | 6468.87 | 53.01 | 6346.82 |
| GROβ 30 min | SEM | 641.1 | 5.25 | 641.1 |
| AMD3100 & | X | 2659.01 | 21.79 | 2536.97 |
| GROβ 60 min | SEM | 226.7 | 1.86 | 226.7 |
| AMD3100 & | X | 2404.16 | 19.70 | 2282.11 |
| GROβ 2.5 hrs | SEM | 333.6 | 2.73 | 333.6 |

TABLE 2
PERIPHERAL BLOOD DIFFERENTIAL SUMMARY

| | WBC | PMN | LYMPH | MONO | RBC | PLT | % PMN | % LYMPH | % MONO |
|---|---|---|---|---|---|---|---|---|---|
| PBS | 8.30 | 1.95 | 5.51 | 0.48 | 6.69 | 759 | 23.4 | 66.4 | 5.9 |
| GROβ 15′ | 11.98 | 3.34 | 7.76 | 0.67 | 9.06 | 800 | 27.35 | 65.19 | 5.65 |
| AMD3100 | 14.49 | 5.35 | 8.54 | 0.47 | 8.56 | 714 | 36.61 | 59.17 | 3.27 |
| 60 min | | | | | | | | | |
| AMD3100 & | 11.69 | 2.30 | 8.25 | 0.76 | 8.40 | 720 | 19.24 | 71.11 | 6.45 |
| GROβ 5 min | | | | | | | | | |
| AMD3100 & | 18.20 | 4.44 | 12.48 | 0.99 | 9.15 | 882 | 24.47 | 68.37 | 5.51 |
| GROβ 15 min | | | | | | | | | |
| AMD3100 & | 21.87 | 8.50 | 12.59 | 1.02 | 8.63 | 803 | 38.99 | 57.92 | 4.69 |
| GROβ 30 min | | | | | | | | | |
| AMD3100 & | 17.53 | 6.84 | 9.77 | 0.62 | 8.28 | 630 | 39.03 | 55.71 | 3.55 |
| GROβ 60 min | | | | | | | | | |
| AMD3100 & | 20.58 | 8.10 | 11.41 | 0.82 | 6.39 | 892 | 39.27 | 55.35 | 4.12 |
| GROβ 2.5 hrs | | | | | | | | | |

As shown in Tables 1 and 2, the combination of AMD3100 plus GROβ acts in an additive to synergistic manner for mobilization of progenitor cells, neutrophils and total white blood cells when compared to either agent alone; response is much more rapid.

It is understood that the foregoing detailed description and accompanying examples are merely illustrative, and are not to be taken as limitations upon the scope of the invention. Various changes and modifications to the disclosed embodiments that are apparent to those skilled in the art may be made without departing from the spirit and scope thereof. U.S. patents and publications referenced herein are incorporated by reference.

1. A method to elevate progenitor and/or stem cell population in peripheral blood or bone marrow which method comprises treating said peripheral blood or bone marrow with a CXCR4 antagonist in combination with a GROβ protein;
in amounts effective to elevate said progenitor and/or stem cell population in said peripheral blood or bone marrow.

2. The method of claim 1, wherein said GROG protein is a modified form OF GROβ protein.

3. The method of claim 2, wherein modified form is SB-251353.

4. The method of claim 1, wherein said CXCR4 antagonist is of the formula
Z-linker-Z′ (1)
or pharmaceutically acceptable salt or prodrug form thereof
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
or Z is of the formula 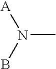
wherein A comprises a monocyclic or bicyclic fused ring system containing at least one N and B is H or an organic moiety of 1-20 atoms,
Z′ may be embodied in a form as defined by Z above, or alternatively may be of the formula
—N(R)—(CR₂)ₙ—X
wherein each R is independently H or straight, branched or cyclic alkyl (1-6C), n is 1 or 2, and X is an aromatic ring, including heteroaromatic rings, or is a mercaptan;
or wherein Z′ can be a nitrogen-containing heterocycle, or NR₂ where each R is as defined above;
“linker” represents a bond, alkylene (1-6C) or may comprise aryl, fused aryl, and/or oxygen atoms contained in an alkylene chain, and/or may contain keto groups and/or nitrogen or sulfur atoms;
or a prodrug or salt thereof.

5. The method of claim 4, wherein at least one of Z and Z′ is a cyclic polyamine.

6. The method of claim 5, wherein Z and Z′ are identical.

7. The method of claim 6, wherein the compound of formula (1) is 1,1′-[1,4-phenylene-bis-(methylene)-bis-1,4,8,11-tetraazacyclotetradecane or a prodrug or salt thereof.

8. The method of claim 4, wherein the compound is
N-[1,4,8,11-tetraazacyclotetradecanyl-1,4-phenylenebis(methylene)]-2-(amino-methyl)pyridine;
N-[1,4,8,11-tetraazacyclotetradecanyl-1,4-phenylenebis(methylene)]-N-methyl-2-(aminomethyl)pyridine;
N-[1,4,8,11-tetraazacyclotetradecanyl-1,4-phenylenebis(methylene)]-4-(amino-methyl)pyridine;
N-[1,4,8,11-tetraazacyclotetradecanyl-1,4-phenylenebis(methylene)]-3-(amino-methyl)pyridine;
N-[1,4,8,11-tetraazacyclotetradecanyl-1,4-phenylenebis(methylene)]-(2-amino-methyl-5-methyl)pyrazine; and
N-[1,4,8,11-tetraazacyclotetradecanyl-1,4-phenylenebis(methylene)]-2-(amino-ethyl)pyridine; or
a prodrug or salt thereof.

9. The method of claim 4, wherein
Z is of the formula 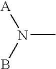
wherein A comprises a monocyclic or bicyclic fused ring system containing at least one N and B is H or an organic moiety of 1-20 atoms.

10. The method of claim 9, wherein the compound of formula (1) is N′-(1H-benzimidazol-2-yl methyl)-N′-(5,6,7,8-tetrahydroquinoline-8-yl)-butane-1,4-diamine,
or a prodrug or salt thereof.

11. The method of claim 4, wherein formula (1) is in the form of its acid addition salt.

12. The method of claim 1, wherein the peripheral blood cells or bone marrow are contained in a living subject.

13. The method of claim 12, wherein the subject exhibits a hematopoietic deficit from chemotherapy or radiation therapy.

14. The method of claim 12, wherein the subject has a condition selected from the group consisting of aplastic anemia, leukemia, drug-induced anemia, neutropenia and thrombocytopenia.

15. The method of claim 12, wherein the subject is a transplantation recipient or is a healthy stem cell donor.

16. The method of claim 12, wherein said progenitor and/or stem cells enhance wound healing, or
ameliorate bacterial inflammation, or
restore damaged cardiac or other organ tissue.

17. The method of claim 16, wherein said progenitor and/or stem cells restore damaged cardiac tissue.

18. The method of claim 12, wherein the CXCR4 antagonist or GROβ protein are administered to said subject by an intravenous or subcutaneous route.

19. The method of claim 12, wherein said CXCR4 antagonist and GROβ protein are administered simultaneously.

20. The method of claim 12, wherein said CXCR4 antagonist and said GROβ protein are administered in tandem.

21. The method of claim 1, wherein said peripheral blood or bone marrow are maintained in culture ex vivo.

22. A combination product comprising a CXCR4 antagonist and a GROβ protein.

23. The combination product of claim 22, wherein said combination is capable of elevating progenitor and/or stem cell population in peripheral blood or bone marrow.

24. A pharmaceutical composition comprising the combination product of claim 22, and a pharmaceutically acceptable excipient.

25. A pharmaceutical composition comprising an effective amount of a CXCR4 antagonist in combination with a GROβ protein.

26. The pharmaceutical composition of claim 25, for elevating progenitor and/or stem cell population in peripheral blood or bone marrow.
